(19) United States
(12) Patent Application Publication
(10) Pub. No.: US 2023/0348465 A1
(43) Pub. Date: Nov. 2, 2023

(54) IBOGAINE ANALOGS AS THERAPEUTICS FOR NEUROLOGICAL AND PSYCHIATRIC DISORDERS

(71) Applicant: The Trustees of Columbia University in the City of New York, New York, NY (US)

(72) Inventors: Dalibor Sames, New York, NY (US); Vaclav Havel, New York, NY (US); Christopher Hwu, Corona, NY (US)

(73) Assignee: The Trustees of Columbia University in the City of New York, New York, NY (US)

(21) Appl. No.: 18/017,000

(22) PCT Filed: Jul. 20, 2021
(86) PCT No.: PCT/US2021/042370

§ 371 Date: Jan. 19, 2023

Related U.S. Application Data
(60) Provisional application No. 63/053,928, filed on Jul. 20, 2020.

(51) Int. Cl. C07D 471/22; B01J 31/02

(52) U.S. Cl. CPC C07D 471/22; B01J 31/0267; C07B 2200/05

(57) ABSTRACT

The present invention provides a compound having the structure:

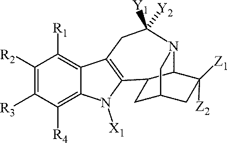

wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
   wherein each Z₃ is, independently, —OH, -O(alkyl), —NH₂, -NH(alkyl) or halogen, and each Z₄ is, independently, —OH, -O(alkyl), —NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, —OAc, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
   wherein each R₅ is, independently, -(alkyl), -(aryl), -(heteroaryl), —OH, -O(alkyl), —NH₂, -NH(alkyl) or -N (alkyl) ₂, and wherein each R₆ is, independently, -(alkyl), -(aryl), -O-(alkyl), -S-(alkyl), -S-(aryl), —NH₂, -NH(alkyl) or -N (alkyl) ₂,
wherein the compound is other than any of ibogaine, ibogamine, N-methyl-ibogaine, N-methyl-noribogaine, N-ethyl-noribogaine, N-methyl-ibogamine or 10-ethoxy-ibogamine,
   or a pharmaceutically acceptable salt of the compound, and methods of using the composition to treat pain, depressive disorders, mood disorders, anxiety disorders, substance use disorders, opioid use disorders, and opioid withdrawal symptoms.

This application claims priority of U.S. Provisional Application No. 63/053,928, filed Jul. 20, 2020, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

Ibogaine is the main indole alkaloid isolated from the root bark of the African shrub Tabernanthe iboga (Alper, K.R. 2001). It is an atypical psychedelic drug capable of inducing waking dream-like states (oneirogenic effects) and vivid memory recall and replay (Naranjo, C. 1973; Brown, T.K. et al. 2019). Anecdotal reports and open label case studies with volunteers dependent on heroin and cocaine indicated ibogaine’s ability to interrupt the drug dependence via rapid and lasting relief of drug withdrawal symptoms and cravings (Alper, K.R. et al. 1999; Mash, D.C. et al. 2018; Schenberg, E.E. et al. 2014). Two recent open label observational clinical studies with subjects diagnosed with opioid dependence confirmed these earlier reports by showing a significant reduction of the withdrawal symptoms (3 days post-treatment) and an improvement of quality of life (up to 12 months) after a single ibogaine therapeutic session (Brown, T.K. et al. 2018; Noller, G.E. et al. 2018). One of these studies also reported a sustained antidepressant effect (evaluated up to 12 months post-treatment) (Noller, G.E. et al. 2018) consistent with the earlier observations of ibogaine’s attenuation of depressive symptoms (Mash, D.C. et al. 2000).

Extensive preclinical work supports the clinical effects of ibogaine in rodent models of substance use disorders (SUDs), including attenuation of self-administration of opioids, cocaine, nicotine, and alcohol, as well as a reduction of opioid withdrawal symptoms in opioid-dependent animals (Glick, S.D. et al. 2001; Belgers, M. et al. 2016). In addition, it has been shown that noribogaine, ibogaine’s major metabolite, exhibits a similar potency and efficacy profile in rodent models of SUDs, leading to the proposal of a mechanistic model where noribogaine contributes to the observed anti-SUD effects (Mash, D.C. et al. 2016).

Considering the large unmet needs in SUDs and psychiatric disorders in general, there is a strong impetus to study biological mechanisms that underpin ibogaine’s effects, and develop new analogs that increase ibogaine’s safety and therapeutic index and enhance our understanding of ibogaine’s mechanism of action. All of these goals are advanced by the design and development of novel analogs of ibogaine and noribogaine.

The present invention is founded on the discovery that small structural modifications of ibogaine or noribogaine have large and unexpected effects on pharmacological activity at monoamine transporters. The modulation of the serotonin transporter (SERT) has been invoked as one of the key molecular mechanisms contributing to ibogaine’s in vivo effects (Baumann, M.H. et al. 2001; Staley, J. K. et al. 1996; Jacobs, M. T. et al. 2007). Furthermore, vesicular monoamine transporter 2 (VMAT2) has been shown as a target for the treatment of SUDs (Nickell, J. R. et al. 2010). VMAT2 inhibitors may also be used to treat hyperkinetic disorders such as Tardive dyskinesia (Solmi, M. 2018; Sreeram, V. et al. 2019), Tourette syndrome (Jankovic, J. et al. 2016) and chorea associated with Huntington’s disease (Dean, M. and Sung, V.W. 2018). We here below show that modulation of VMAT2 contributes to ibogaine’s effects, and further show that for some compounds dual inhibition of SERT and VMAT2 is mechanistically important. Furthermore, the data herein shows that some compounds selectively inhibit VMAT2, while other compounds selectively inhibit SERT, and still other compounds inhibit both VMAT2 and SERT.

The data here also shows the unexpected effect of 10-ethoxy-ibogamine on monoamine transporters. 10-Ethoxyibogamine is a potent VMAT2 inhibitor but is only a weak inhibitor of SERT. In contrast, noribogaine, which can be formed by the metabolism of 10-ethoxy-ibogamine, is a potent SERT inhibitor. Therefore, we can achieve dual modulation of VMAT2 and SERT through a combined effect of the administered drug and its metabolite.

### SUMMARY OF THE INVENTION

The present invention provides a compound having the structure:

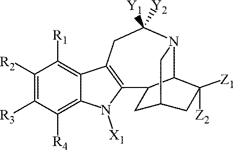

wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
   wherein each Z₃ is, independently, —OH, -O(alkyl), —NH₂, -NH(alkyl) or halogen, and each Z₄ is, independently, —OH, -O(alkyl), —NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, —OAc, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₄, -SC(O)R₅, -NHC(O)R₆ or -NHC(S)R₆,
   wherein each R₅ is, independently, -(alkyl), -(aryl), -(heteroaryl), —OH, -O (alkyl), —NH₂, -NH(alkyl) or -N (alkyl) ₂, and
   wherein each R₆ is, independently, -(alkyl), -(aryl), -O-(alkyl), -S-(alkyl), -S-(aryl), —NH₂, -NH(alkyl) or -N (alkyl) ₂,
wherein the compound is other than any of ibogaine, ibogamine, N-methyl-ibogaine, N-methyl-noribogaine, N-ethyl-noribogaine, N-methyl-ibogamine or 10-ethoxy-ibogamine,
or a pharmaceutically acceptable salt of the compound.

The present invention also provides a method of inhibiting serotonin transporter (SERT) and/or vesicular monoamine transporter 2 (VMAT2) in a subject comprising administering to the subject an effective amount of a compound having the structure:

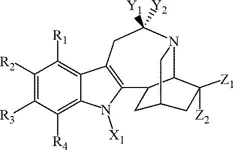

wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
   wherein each Z₃ is, independently, —OH, -O (alkyl), —NH₂, -NH (alkyl) or halogen, and each Z₄ is, independently, —OH, -O(alkyl), —NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, -OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
   wherein each R₅ is, independently, -(alkyl), -(aryl), -(heteroaryl), —OH, -O(alkyl), —NH₂, -NH(alkyl) or -N (alkyl) ₂, and
   wherein each R₆ is, independently, -(alkyl), -(aryl), -O-(alkyl), -S-(alkyl), -S-(aryl), —NH₂, -NH(alkyl) or -N (alkyl) ₂,
or a pharmaceutically acceptable salt thereof, so as to thereby inhibit serotonin transporter (SERT) and/or vesicular monoamine transporter 2 (VMAT2) in the subject.

### BRIEF DESCRIPTION OF THE FIGURES

FIG. 1: Select Ibogaine Analogs

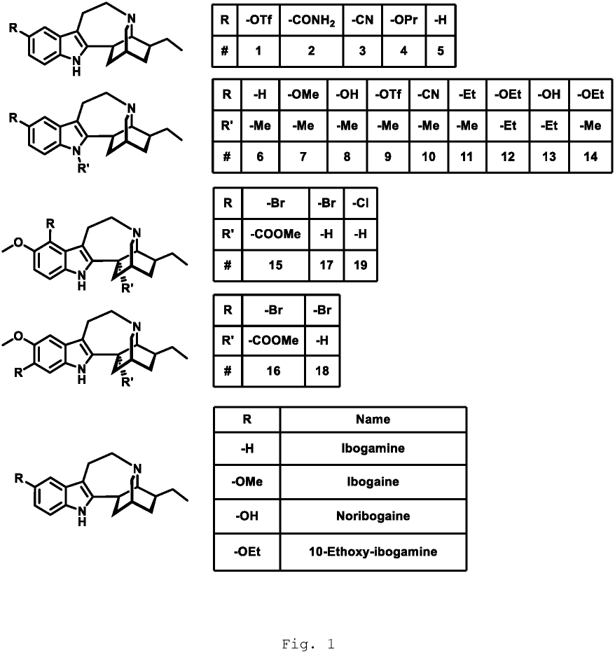

FIG. 2: Inhibition of SERT by Noribogaine and Compounds 5, 8, and 17 using the fluorescent substrate, APP⁺. Curves represent the average values of experiments n ≥ 4, with error bars signifying ± SEM.

FIG. 3: Inhibition of VMAT2 by Noribogaine, 10-Ethoxy-ibogamine, and Compounds 5, 8, and 17 using the fluorescent substrate, FFN206. Curves represent the average values of experiments n ≥ 4, with error bars signifying ± SEM.

FIG. 4: Conversion of 10-Ethoxy-ibogamine to Noribogaine in rat liver microsomes.

FIG. 5: Conversion of 10-Ethoxy-ibogamine to Noribogaine in human liver microsomes.

### DETAILED DESCRIPTION OF THE INVENTION

The present invention provides a compound having the structure:

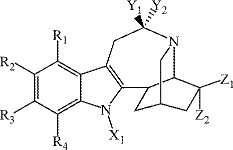

wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
   wherein each Z₃ is, independently, —OH, -O(alkyl), —NH₂, -NH(alkyl) or halogen, and each Z₄ is, independently, —OH, -O(alkyl), —NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
   wherein each R₅ is, independently, -(alkyl), -(aryl), -(heteroaryl), —OH, -O(alkyl), —NH₂, -NH(alkyl) or -N(alkyl)₂, and
   wherein each R₆ is, independently, -(alkyl), -(aryl), -O-(alkyl), -S-(alkyl), -S-(aryl), —NH₂, -NH(alkyl) or -N (alkyl) ₂,
or a pharmaceutically acceptable salt of the compound.

In some embodiments of the above compound,

when X₁ is H, then R₁, R₂, R₃ and R₄ are each, independently, —F, —NO₂, —CN, —CF₃, —CF₂H, —OCF₃, - (alkyl), - (alkenyl), - (alkynyl), - (aryl), -(heteroaryl), -O-(alkenyl), -O-(alkynyl), -O-(aryl), -O-(heteroaryl), -S-(alkyl), -S-(alkenyl), S-(alkynyl), -S-(aryl), -S-(heteroaryl), -C (O) R₅, -S (O) R₅, -SO₂R₅, -NHSO₂R₅, -SC (O) R₅, -NHC(O)R₆ or -NHC(S)R₆,
wherein each R₅ is, independently, -(alkyl), -(aryl), -(heteroaryl), —OH, -O (alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂,

In some embodiments of the above compound,

when X₁ is H, then R₂ is —F, —NO₂, —CN, —CF₃, —CF₂H, —OCF₃, - (alkyl), -(alkenyl), -(alkynyl), -(aryl), -(heteroaryl), -O-(alkenyl), -O-(alkynyl), -O-(aryl), -O-(heteroaryl), -S-(alkyl), -S-(alkenyl), S-(alkynyl), -S- (aryl), -S- (heteroaryl), -C(O)R₅, -S(O)R₅, -SO₂R₅, -NHSO₂R₅, -SC(O)R₅, -NHC(O)R₆ or -NHC(S)R₆,
wherein each R₅ is, independently, -(alkyl), -(aryl), -(heteroaryl), —OH, —O (alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂,

In some embodiments of the above compound,

when X₁, Y₁, Y₂, Z₂, R₁, R₃ and R₄ are each H and Z₁ is ethyl, then R₂ is —F, —NO₂, —CN, —CF₃, —CF₂H, —OCF₃, - (alkyl), - (alkenyl), - (alkynyl), -(aryl), -(heteroaryl), -O-(alkenyl), -O-(alkynyl), -O-(aryl), -O-(heteroaryl), -S-(alkyl), -S-(alkenyl), S-(alkynyl), -S-(aryl), -S-(heteroaryl), -C (O) R₅, -S (O) R₅, -SO₂R₅, -NHSO₂R₅, -SC (O) R₅, -NHC(O)R₆ or -NHC (S)R₆,
wherein each R₅ is, independently, -(alkyl), -(aryl), -(heteroaryl), —OH, -O(alkyl), —NH₂, -NH(alkyl) or -N(alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂,

In some embodiments of the above compound,

when X₁ is alkyl, then R₂ is —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH- (aryl), -NH- (heteroaryl), -C (O) R₅, -S (O) R₅, -SO₂R₅, -NHSO₂R₅, -OC(O)R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N(alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂,

In some embodiments of the above compound,
one of R₁, R₃ and R₄ is —F, —Cl, —Br, —I, —NO₂, —CN, —CF₃, —CF₂H, —OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH- (heteroaryl), -C(O)R₅, -S(O)R₅, -SO₂R₅, -NHSO₂R₅, -OC(O)R₅, -SC(O)R₅, -NHC(O)R₆ or -NHC(S)R₆,
   wherein each R₅ is, independently, -(alkyl), -(aryl), -(heteroaryl), —OH, -O(alkyl), —NH₂, -NH(alkyl) or -N(alkyl) ₂, and
   wherein each R₆ is, independently, -(alkyl), -(aryl), -O-(alkyl), -S-(alkyl), -S-(aryl), —NH₂, -NH(alkyl) or -N(alkyl) ₂, and
the others are —H; and
R₂ is —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC (O) R₆ or -NHC(S) R₆,
   wherein each R₅ is, independently, -(alkyl), -(aryl), -(heteroaryl), —OH, -O(alkyl), —NH₂, -NH(alkyl) or -N(alkyl) ₂, and
   wherein each R₆ is, independently, -(alkyl), -(aryl), -O-(alkyl), -S-(alkyl), -S-(aryl), —NH₂, -NH(alkyl) or -N (alkyl) ₂.

In certain embodiments the compound has the structure below wherein the substituents are defined as in the preceding paragraph:

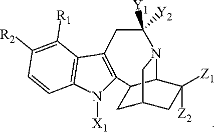

In certain embodiments the compound has the structure below wherein the substituents are defined as in the paragraph above:

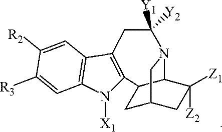

In another embodiment of the invention provides any of the structures wherein
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, -O(alkyl), —NH₂, -NH(alkyl) or halogen, and each Y₄ is, independently, -O(alkyl), —NH₂, -NH(alkyl) or -N(alkyl)₂.

In some embodiments, the compound wherein
one of Y₁ and Y₂ is -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, -O(alkyl), —NH₂, -NH(alkyl) or halogen, and each Y₄ is, independently-O(alkyl), —NH₂, -NH(alkyl) or -N(alkyl)₂, and
the other is —H.

In some embodiments, the compound wherein
one of Y₁ and Y₂ is -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂, and
the other is —H.

In some embodiments, the compound wherein Y₁ and Y₂ are each H. In some embodiments, the compound wherein Z₁ is —CH₂CH₃ and Z₂ is —H.

In some embodiments, the compound wherein Z₁ is —CH₂CH₃; Z₂ is —H; and X₁ is —H.

In some embodiments, the compound wherein Z₁ is —CH₂CH₃; Z₂ is —H; and X₁ is -(alkyl).

In some embodiments, the compound wherein one of X₁, Y₁, Y₂, Z₂, R₁, R₃ and R₄ is other than —H.

In some embodiments, the compound wherein two of X₁, Y₁, Y₂, Z₂, R₁, R₃ and R₄ are other than —H.

In some embodiments, the compound wherein three of X₁, Y₁, Y₂, Z₂, R₁, R₃ and R₄ are other than —H.

In some embodiments, the compound wherein four of X₁, Y₁, Y₂, Z₂, R₁, R₃ and R₄ are other than —H.
In an embodiment wherein R₂ is -OCH₃, X₁ is —CH₃, Z₁ is —CH₂CH₃ than one of R₁ or R₃ or R₄ is other than —H,
In an embodiment wherein R₂ is —OH, X₁ is —CH₃, Z₁ is —CH₂CH₃ than one of R₁ or R₃ or R₄ is other than —H,
In an embodiment wherein R₂ is —OH, X₁ is —CH₂CH₃, Z₁ is —CH₂CH₃ than one of R₁ or R₃ or R₄ is other than —H,
In an embodiment wherein R₂ is —H, X₁ is —CH₃, Z₁ is —CH₂CH₃ than one of R₁ or R₃ or R₄ is other than —H,
In an embodiment wherein R₂ is -OCH₂CH₃, X₁ is —H, Z₁ is —CH₂CH₃ than one of R₁ or R₃ or R₄ is other than —H,
In some embodiments, the compound having the structure:

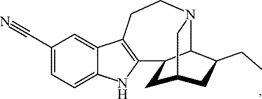

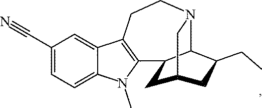

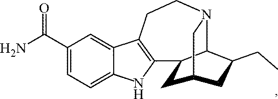

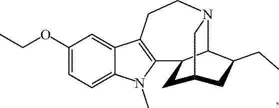

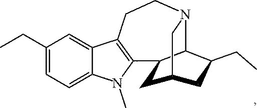

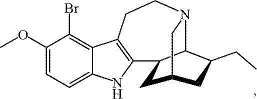

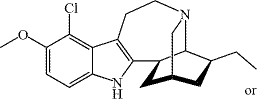

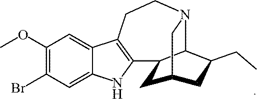

In some embodiments, the compound wherein R₂ is —OCH₃ or —OCH₂CH₃.

In some embodiments, the compound wherein R₂ is

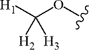

wherein at least one of H₁, H₂ or H₃ is a deuterium-enriched -H site, or

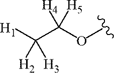

wherein at least one of H₁, H₂, H₃, H₄ or H₅ is a deuterium-enriched —H site.

The present invention provides a composition which comprises a compound which is a mixture of deuterium containing and non-deuterium containing molecules having the structure of the present invention or a pharmaceutically acceptable salt of the compound, wherein in the mixture the proportion of molecules having deuterium at least one of H₁, H₂, H₃ H₄ or H₅ position is substantially greater than 0.0156% of molecules in the mixture.

The present invention provides a composition which comprises a carrier and a compound having the structure of the present invention or a pharmaceutically acceptable salt of the compound.

In some embodiments, wherein R₂ is

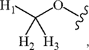

wherein at least one of H₁, H₂ or H₃ is a deuterium-enriched —H site.

In some embodiments, wherein each of H₁-H₃ are deuterium-enriched.

In some embodiments, wherein the proportion of molecules having deuterium at each of the H₁-H₃ positions is substantially greater than 90% of molecules in the composition.

In some embodiments, wherein two of H₁-H₃ are deuterium-enriched.

In some embodiments, wherein the proportion of molecules having deuterium at two of the H₁-H₃ positions is substantially greater than 90% of molecules in the composition.

In some embodiments, wherein one of H₁-H₃ is deuterium-enriched.

In some embodiments, wherein the proportion of molecules having deuterium at one of the H₁-H₃ positions is substantially greater than 90% of molecules in the composition.

In some embodiments, wherein R₂ is

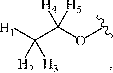

wherein at least one of H₁, H₂, H₃, H₄ or H₅ is a deuterium-enriched —H site.

In some embodiments, wherein each of H₁-H₅ are deuterium-enriched.

In some embodiments, wherein the proportion of molecules having deuterium at each of the H₁-H₅ positions is substantially greater than 90% of molecules in the composition.

In some embodiments, wherein each of H₁-H₅ are deuterium-enriched.

In some embodiments, wherein the proportion of molecules having deuterium at each of the H₁-H₅ positions is substantially greater than 90% of molecules in the composition.

In some embodiments, wherein each of H₄-H₅ are deuterium-enriched or one of H₄-H₅ is deuterium-enriched.

In some embodiments, wherein the proportion of molecules having deuterium at each of the H₄-H₅ positions is substantially greater than 90% of molecules in the composition, or the proportion of molecules having deuterium at one of the H₄-H₅ positions is substantially greater than 90% of molecules in the composition.

In some embodiments, wherein R₂ is

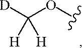

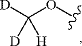

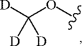

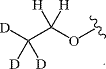

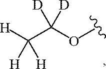

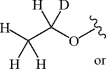

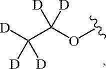

wherein D represents a deuterium-enriched —H site.

In some embodiments, wherein R₁ is

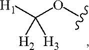

wherein at least one of H₁, H₂ or H₃ is a deuterium-enriched —H site.

In some embodiments, wherein each of H₁-H₃ are deuterium-enriched. In some embodiments, wherein two of H₁-H₃ are deuterium-enriched.

In some embodiments, wherein one of H₁-H₃ is deuterium-enriched.

In some embodiments, wherein R₁ is

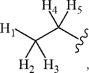

wherein at least one of H₁, H₂, H₃, H₄ or H₅ is a deuterium-enriched —H site.

In some embodiments, wherein each of H₁-H₅ are deuterium-enriched or each of H₁-H₃ are deuterium-enriched.

In some embodiments, wherein each of H₄-H₅ are deuterium-enriched or one of H₄-H₅ is deuterium-enriched.

In some embodiments, wherein R₂ is

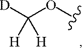

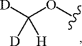

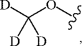

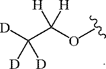

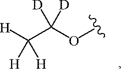

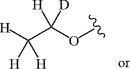

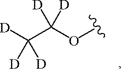

wherein D represents a deuterium-enriched —H site.

In some embodiments, the composition further comprising a carrier.

In some embodiments, wherein the carrier is a pharmaceutically acceptable carrier.

The present invention provides a pharmaceutical composition comprising the compound of the present invention and a pharmaceutically acceptable carrier.

In some embodiments, the composition further comprising a mu-opioid receptor agonist.

In some embodiments, the composition further comprising an opioid or opiate.

In some embodiments, wherein the opioid or opiate is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]18hiazepine-11-yl)amino)heptanoic acid, 7-((3-iodo-6-methyl-5,5-dioxido-6,11-dihydrodibenzo[c,f] [1,2] 18hiazepine-11-yl)amino)heptanoic acid, 5-((3-bromo-6-methyl-5,5-dioxido-6,11-dihydrodibenzo[c,f] [1,2]18hiazepine-11-yl)amino)pentanoic acid or 5-((3-iodo-6-methyl-5,5-dioxido-6,11-dihydrodibenzo[c,f] [1,2]18hiazepine-11-yl)amino)pentanoic acid.

The present invention provides a method of altering the psychological state of a subject comprising administering to the subject the compound of the present invention, or the composition of the present invention comprising an effective amount of the compound, so as to thereby alter the psychological state of the subject.

The present invention provides a method of enhancing the effect of psychotherapy in a subject comprising administering to the subject the compound of the present invention, or the composition of the present invention comprising an effective amount of the compound, so as to thereby enhance the effect of the psychotherapy in the subject.

The present invention provides a method of inducing wakefulness or decreasing sleepiness in a subject comprising administering to the subject the compound of the present invention, or the composition of the present invention comprising an effective amount of the compound, so as to thereby induce wakefulness or decrease sleepiness in the subject.

The present invention provides a method of decreasing the duration of REM sleep in a subject comprising administering to the subject the composition of the present invention comprising an effective amount of the compound so as to thereby decrease the duration of REM sleep in the subject.

The present invention provides a method of increasing energetic feelings in a subject comprising administering to the subject the composition of the present invention comprising an effective amount of the compound so as to thereby increase the energetic feelings in the subject.

The present invention provides a method of inducing a stimulating effect in a subject comprising administering to the subject the compound of the present invention, or the composition of the present invention comprising an effective amount of the compound, so as to thereby induce the stimulating effect in the subject.

In some embodiments, the stimulating effect is a central stimulating effect.

In some embodiments, the stimulating effect is induced substantially free of undesired side-effects in the subject.

In some embodiments, the stimulating effect is induced without inducing an addictive effect in the subject to the compound.

In some embodiments, a use of the composition of the present invention comprising an effective amount of the compound as a stimulant.

The present invention provides a method of treating a subject afflicted with substance use disorder comprising administering to the subject the compound of the present invention, or the composition of the present invention comprising an effective amount of the compound, so as to thereby treat the subject afflicted with the substance use disorder.

In some embodiments, wherein the substance use disorder is opioid use disorder, alcohol use disorder or stimulant use disorder.

The present invention provides a method of treating a subject afflicted with opioid withdrawal symptoms comprising administering to the subject the compound of the present invention, or the composition of the present invention comprising an effective amount of the compound, so as to thereby treat the subject afflicted with the opioid withdrawal symptoms.

The present invention provides a method of treating a subject afflicted with a depressive disorder, a mood disorder, an anxiety disorder, Parkinson’s disease, or traumatic brain injury comprising administering to the subject the compound of the present invention, or the composition of the present invention comprising an effective amount of the compound, so as to thereby treat the subject afflicted with the depressive disorder, the mood disorder, the anxiety disorder, Parkinson’s disease or the traumatic brain injury.

The present invention provides a method of treating a subject afflicted with pain comprising administering to the subject the composition of the present invention comprising an effective amount of the compound and the opioid or opiate so as to thereby treat the subject afflicted with pain.

In some embodiments, wherein an effective amount of 10-1500 mg of the compound is administered to the subject.

The present invention provides a method of inhibiting serotonin transporter (SERT) and vesicular monoamine transporter 2 (VMAT2) in a subject comprising administering to a subject an effective amount of a compound having the structure:

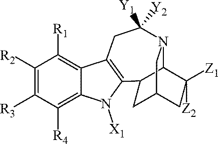

wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
   wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
   wherein each Z₃ is, independently, —OH, -O(alkyl), —NH₂, -NH(alkyl) or halogen, and each Z₄ is, independently, —OH, -O(alkyl), —NH₂, -NH(alkyl) or -N(alkyl)₂;
R₁, R₂, R₃, and R₄ are each, independently, —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, -Oac, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
   wherein each R₅ is, independently, -(alkyl), -(aryl), -(heteroaryl), —OH, -O(alkyl), —NH₂, -NH(alkyl) or -N(alkyl)₂, and
   wherein each R₆ is, independently, -(alkyl), -(aryl), -O-(alkyl), -S-(alkyl), -S-(aryl), —NH₂, -NH(alkyl) or -N (alkyl) ₂,
or a pharmaceutically acceptable salt thereof, so as to thereby inhibit serotonin transporter (SERT) and vesicular monoamine transporter 2 (VMAT2) in a subject.

In some embodiments of any of the above methods, the compound inhibits serotonin transporter (SERT) and vesicular monoamine transporter 2 (VMAT2) or any combination thereof in a subject.

In some embodiments of any of the above methods, the compound inhibits serotonin transporter (SERT) in a subject.

In some embodiments of any of the above methods, the compound inhibits vesicular monoamine transporter 2 (VMAT2) in a subject.

In some embodiments of any of the above methods, the compound inhibits serotonin transporter (SERT) and vesicular monoamine transporter 2 (VMAT2).

The present invention provides a method of altering the psychological state of a subject by inhibiting serotonin transporter (SERT) and/or vesicular monoamine transporter 2 (VMAT2) in a subject.

The present invention provides a method of enhancing the effect of psychotherapy in a subject by inhibiting serotonin transporter (SERT) and/or vesicular monoamine transporter 2 (VMAT2) in a subject.

The present invention provides a method of inducing wakefulness or decreasing sleepiness in a subject by inhibiting serotonin transporter (SERT) and/or vesicular monoamine transporter 2 (VMAT2) in a subject.

The present invention provides a method of inducing a stimulating effect in a subject by inhibiting serotonin transporter (SERT) and/or vesicular monoamine transporter 2 (VMAT2) in a subject.

The present invention provides a method of treating a subject afflicted with substance use disorder by inhibiting serotonin transporter (SERT) and/or vesicular monoamine transporter 2 (VMAT2) in a subject.

The present invention provides a method of treating a subject afflicted with opioid use disorder by inhibiting serotonin transporter (SERT) and/or vesicular monoamine transporter 2 (VMAT2) in a subject.

The present invention provides a method of treating a subject afflicted with alcohol use disorder by inhibiting serotonin transporter (SERT) and/or vesicular monoamine transporter 2 (VMAT2) in a subject.

The present invention provides a method of treating a subject afflicted with stimulant use disorder by inhibiting serotonin transporter (SERT) and/or vesicular monoamine transporter 2 (VMAT2) in a subject.

The present invention provides a method of treating a subject afflicted with opioid withdrawal symptoms by inhibiting serotonin transporter (SERT) and/or vesicular monoamine transporter 2 (VMAT2) in a subject.

The present invention provides a method of treating a subject afflicted with a depressive disorder, a mood disorder, an anxiety disorder, Parkinson’s disease, or traumatic brain injury by inhibiting serotonin transporter (SERT) and/or vesicular monoamine transporter 2 (VMAT2) in a subject.

The present invention provides a method of inhibiting vesicular monoamine transporter 2 (VMAT2), or inhibiting both VMAT2 and SERT in a subject.

The present invention provides a method of treating a subject afflicted with substance use disorder by inhibiting vesicular monoamine transporter 2 (VMAT2), or inhibiting both VMAT2 and SERT in a subject.

The present invention provides a method of treating a subject afflicted with Tardive dyskinesia (TD) by inhibiting vesicular monoamine transporter 2 (VMAT2), or inhibiting both VMAT2 and SERT in a subject.

The present invention provides a method of treating a subject afflicted with Tourette syndrome by inhibiting vesicular monoamine transporter 2 (VMAT2), or inhibiting both VMAT2 and SERT in a subject.

The present invention provides a method of treating a subject afflicted with chorea associated with Huntington’s disease by inhibiting vesicular monoamine transporter 2 (VMAT2), or inhibiting both VMAT2 and SERT in a subject.

In some embodiments, wherein an effective amount of 10-1500 mg of the compound is administered to the subject.

In some embodiments of the present method, the compound has the structure:

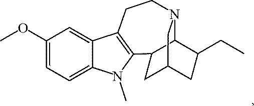

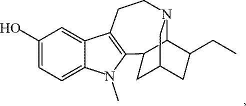

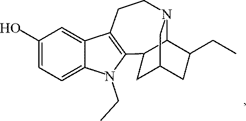

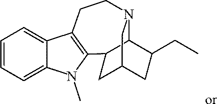

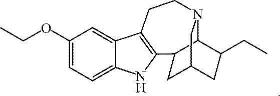

The present invention further provides a process for producing the compound having the structure:

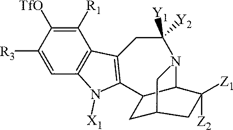

comprising
(a) contacting a compound having the structure:

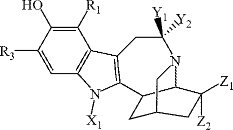

with bis(trifluoromethanesulfonyl)aniline in a first suitable solvent; and
(b) adding a base to produce the compound having the structure:

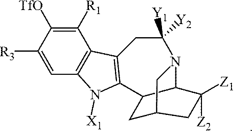

In some embodiments, the process wherein the first suitable solvent is dichloroethane.

In some embodiments, the process wherein the base is triethylamine.

The present invention further provides a process for producing the compound having the structure:

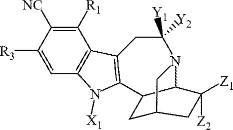

comprising contacting a compound having the structure:

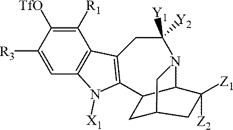

with a preformed palladium (0) catalyst in the presence of ZnCN₂ in a first suitable solvent to produce the compound having the structure:

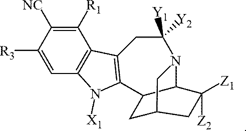

In some embodiments, the process wherein the palladium (0) catalyst is Pd(PPh₃)₄.

In some embodiments, the process wherein the first suitable solvent is DMF.

The present invention further provides a process for producing the compound having the structure:

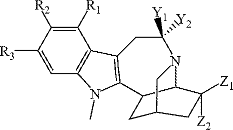

comprising
(a) contacting a compound having the structure:

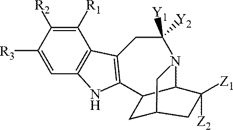

with a base in a first suitable solvent;
(b) adding a methylating reagent to produce the compound having the structure:

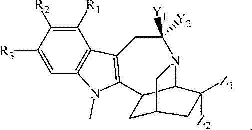

In some embodiments, the process wherein the first suitable solvent is DMSO.

In some embodiments, the process wherein the base is potassium hydroxide.

In some embodiments, the process wherein the methylating reagent is iodomethane.

The present invention further provides a process for producing the compound having the structure:

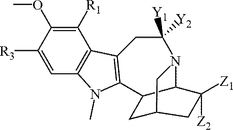

comprising
(a) contacting a compound having the structure:

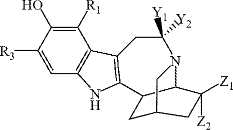

with a base in a first suitable solvent; and
(b) adding a methylating reagent to produce the compound having the structure:

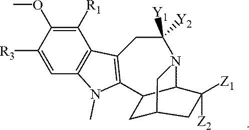

In some embodiments, the process wherein the first suitable solvent is DMSO.

In some embodiments, the process wherein the base is potassium hydroxide.

In some embodiments, the process wherein the methylating reagent is iodomethane.

The present invention further provides a process for producing the compound having the structure:

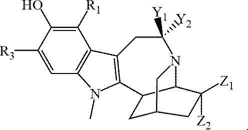

comprising
contacting a compound having the structure:

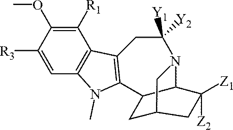

with BBr₃ and a nucleophile in a first suitable solvent to produce the compound having the structure:

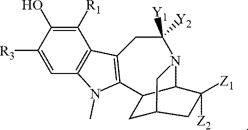

In some embodiments, the process wherein the first suitable solvent is dichloromethane.

In some embodiments, the process wherein the nucleophile is a soft nucleophile.

In some embodiments, the process wherein the soft nucleophile is EtSH.

The present invention further provides a process for producing the compound having the structure:

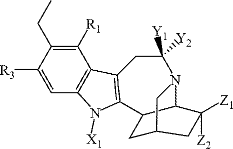

comprising
(a) contacting a compound having the structure:

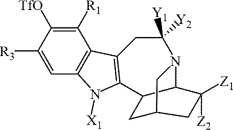

with a palladium (0) reagent in the presence of a ligand, base and CH₃CH₂BF₃K.
(b) adding a first suitable solvent mixture to produce the compound having the structure:

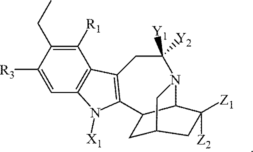

In some embodiments, the process wherein the palladium (0) reagent is Pd(Oac)₂.

In some embodiments, the process wherein the ligand is RuPhos.

In some embodiments, the process wherein the base is cesium carbonate.

In some embodiments, the process wherein the first suitable solvent mixture is toluene and water.

The term “ibogaine” refers to the structure:

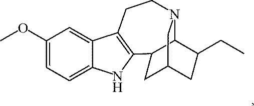

The term “ibogamine” refers to the structure:

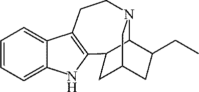 [SMILES: CCC1CC2CC3c4[nH]c5ccccc5c4CCN(C2)C13]

The term “N-methyl-ibogaine” refers to the structure:

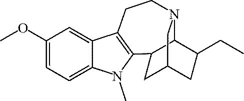 [SMILES: CCC1CC2CC3c4c(c5cc(OC)ccc5n4C)CCN(C2)C13]

The term “N-methyl-noribogaine” refers to the structure:

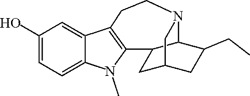 [SMILES: CCC1CC2CC3c4c(c5cc(O)ccc5n4C)CCN(C2)C13]

The term “N-ethyl-noribogaine” refers to the structure:

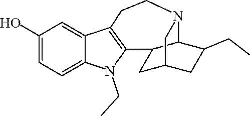 [SMILES: CCC1CC2CC3c4c(c5cc(O)ccc5n4CC)CCN(C2)C13]

The term “N-methyl-ibogamine” refers to the structure:

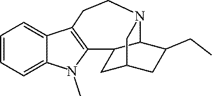 [SMILES: CCC1CC2CC3c4c(c5ccccc5n4C)CCN(C2)C13]

The term “10-ethoxy-ibogamine” refers to the structure:

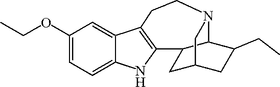 [SMILES: CCOc1ccc2[nH]c3c(c2c1)CCN1CC2CC(CC)C1C3C2]

In some embodiments of any of the above composition, the composition further comprising a carrier.

In some embodiments of any of the above composition, the composition wherein the carrier is a pharmaceutically acceptable carrier.

In some embodiments of any of the above compositions, the composition further comprising a mu-opioid receptor agonist.

In some embodiments of any of the above compositions, the composition further comprising an opioid or opiate.

In some embodiments of any of the above compositions, the composition further comprising morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]32hiazepine-11-yl)amino)heptanoic acid, 7-((3-iodo-6-methyl-5,5-dioxido-6,11-dihydrodibenzo[c,f][1,2] 32hiazepine-11-yl)amino)heptanoic acid, 5-((3-bromo-6-methyl-5,5-dioxido-6,11-dihydrodibenzo[c,f][1,2]32hiazepine-11-yl)amino)pentanoic acid or 5-((3-iodo-6-methyl-5,5-dioxido-6,11-dihydrodibenzo[c,f][1,2]32hiazepine-11-yl)amino)pentanoic acid.

In some embodiments of any of the above compositions, the composition further comprising any of the compounds disclosed in PCT International Publication Nos. WO 2015/138791, WO 2017/049158, WO 2018/170275 or WO 2020/037136, the contents of each of which are hereby incorporated by reference.

In some embodiments, a method of altering the psychological state of a subject comprising administering to the subject the composition of the present invention comprising an effective amount of the compound so as to thereby alter the psychological state of the subject.

In some embodiments, a method of enhancing the effect of psychotherapy comprising administering to the subject the composition of the present invention comprising an effective amount of the compound so as to thereby enhance the effect of the psychotherapy.

In some embodiments, a method of treating a subject afflicted with a depressive disorder, a mood disorder or an anxiety disorder, comprising administering to the subject the composition of the present invention comprising an effective amount of the compound so as to thereby treat the subject afflicted with the depressive disorder, the mood disorder or the anxiety disorder.

In some embodiments, the depressive disorder, the mood disorder, or the anxiety disorder.

In some embodiments, a method of reducing opioid cravings in a subject afflicted with an opioid use disorder comprising administering to the subject the composition of the present invention comprising an effective amount of the compound so as to reduce the subject’s opioid cravings.

In some embodiments, a method of treating a subject afflicted with a substance use disorder comprising administering to the subject the composition of the present invention comprising an effective amount of the compound so as to treat the subject afflicted with the substance use disorder.

In some embodiments, wherein the substance use disorder is opioid use disorder, alcohol use disorder or stimulant use disorder.

In some embodiments, wherein the substance use disorder is opioid use disorder, alcohol use disorder, stimulant use disorder or polydrug use disorder.

In some embodiments, wherein the stimulant use disorder is nicotine use disorder.

In some embodiments, a method of treating a subject afflicted with opioid withdrawal symptoms comprising administering to the subject the composition of the present invention comprising an effective amount of the compound so as to treat the subject afflicted with the opioid withdrawal symptoms.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with alcohol withdrawal symptoms or stimulant withdrawal symptoms comprising administering to the subject the composition of the present invention comprising an effective amount of the compound so as to treat the subject afflicted with the opioid withdrawal symptoms.

In some embodiments, a method of treating a subject afflicted with traumatic brain injury (TBI) comprising administering to the subject the composition of the present invention comprising an effective amount of the compound so as to treat the subject afflicted with the traumatic brain injury (TBI).

In some embodiments, a method of treating a subject afflicted with Parkinson’s disease comprising administering to the subject the composition of the present invention comprising an effective amount of the compound so as to treat the subject afflicted with the Parkinson’s disease.

In some embodiments, a method of treating a subject afflicted with opioid use disorder comprising administering to the subject an effective amount of mu-opioid receptor agonist and the composition of the present invention comprising an effective amount of the compound so as to treat the subject afflicted with the opioid use disorder.

In some embodiments, a method of treating a subject afflicted with pain comprising administering to the subject an effective amount of an opioid or opiate and the composition of the present invention comprising an effective amount of the compound so as to treat the subject afflicted with pain.

In some embodiments, a method of treating a subject afflicted with pain comprising administering to the subject an effective amount of morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepine-11-yl)amino)heptanoic acid, 7-((3-iodo-6-methyl-5,5-dioxido-6,11-dihydrodibenzo[c,f] [1,2]thiazepine-11-yl)amino)heptanoic acid, 5-((3-bromo-6-methyl-5,5-dioxido-6,11-dihydrodibenzo[c,f][1,2]thiazepine-11-yl)amino)pentanoic acid or 5-((3-iodo-6-methyl-5,5-dioxido-6,11-dihydrodibenzo[c,f] [1,2]thiazepine-11-yl)amino)pentanoic acid and the composition of the present invention comprising an effective amount of the compound so as to treat the subject afflicted with pain.

In some embodiments, a method of treating a subject afflicted with opioid use disorder comprising administering to the subject an effective amount of an opioid or opiate and the composition of the present invention comprising an effective amount of the compound so as to treat the subject afflicted with the opioid use disorder.

In some embodiments, a method of treating a subject afflicted with opioid use disorder comprising administering to the subject an effective amount of morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepine-11-yl)amino)heptanoic acid, 7-((3-iodo-6-methyl-5,5-dioxido-6,11-dihydrodibenzo[c,f] [1,2]thiazepine-11-yl)amino)heptanoic acid, 5-((3-bromo-6-methyl-5,5-dioxido-6,11-dihydrodibenzo[c,f][1,2]thiazepine-11-yl)amino)pentanoic acid or 5-((3-iodo-6-methyl-5,5-dioxido-6,11-dihydrodibenzo[c,f] [1,2]thiazepine-11-yl)amino)pentanoic acid and the composition of the present invention comprising an effective amount of the compound so as to treat the subject afflicted with the opioid use disorder.

In some embodiments, a method of treating a subject afflicted with opioid use disorder or opioid withdrawal symptoms comprising administering to the subject an effective amount of naloxone or methylnaltrexone and the composition of the present invention comprising an effective amount of the compound so as to thereby treat the subject afflicted with the opioid use disorder or opioid withdrawal symptoms.

The present invention also provides a compound having the structure:

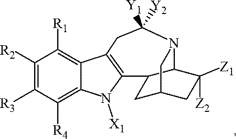

or a salt thereof, for use in treating a subject afflicted with a depressive disorder, a mood disorder, an anxiety disorder, a substance use disorder, opioid withdrawal symptoms, traumatic brain injury, or Parkinson’s disease.

The present invention also provides a compound having the structure:

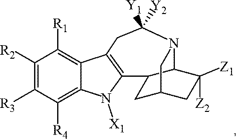

or a salt thereof, for use in treating a subject afflicted with a depressive disorder, a mood disorder, an anxiety disorder, a substance use disorder, opioid withdrawal symptoms, traumatic brain injury, or Parkinson’s disease.

The present invention further provides a pharmaceutical composition comprising an amount of a compound having the structure:

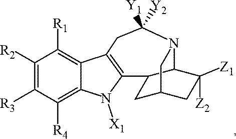

or a salt thereof, for use in treating a subject afflicted with a depressive disorder, a mood disorder, an anxiety disorder, a substance use disorder, opioid withdrawal symptoms, traumatic brain injury, or Parkinson’s disease.

The present invention also provides a compound having the structure:

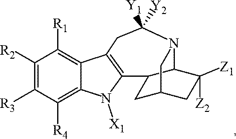

or a salt thereof, for use as an add-on therapy or in combination with an opioid or opiate in treating a subject afflicted with pain, adepressive disorder, a mood disorder, an anxiety disorder, a substance use disorder, opioid withdrawal symptoms, traumatic brain injury, or Parkinson’s disease.

In some embodiments, a package comprising:
a) a first pharmaceutical composition comprising an amount of an opioid or opiate and a pharmaceutically acceptable carrier;
b) a second pharmaceutical composition comprising the compound of the present invention and a pharmaceutically acceptable carrier; and
c) instructions for use of the first and second pharmaceutical compositions together to treat a subject afflicted with pain, a depressive disorder, a mood disorder, an anxiety disorder, a substance use disorder, opioid withdrawal symptoms, traumatic brain injury, or Parkinson’s disease.

In some embodiments, a therapeutic package for dispensing to, or for use in dispensing to, a subject afflicted pain, a depressive disorder, a mood disorder, an anxiety disorder, a substance use disorder, opioid withdrawal symptoms, traumatic brain injury or Parkinson’s disease, which comprises:
a) one or more unit doses, each such unit dose comprising:
   (i) a pharmaceutical composition comprising the compound of the present invention; and
   (ii) an amount of an opioid or opiate,
      wherein the respective amounts of said composition and said opioid or opiate in said unit dose are effective, upon concomitant administration to said subject, to treat the subject, and
(b) a finished pharmaceutical container therefor, said container containing said unit dose or unit doses, said container further containing or comprising labeling directing the use of said package in the treatment of said subject.

The therapeutic package of the above embodiment, wherein the respective amounts of said composition and opioid or opiate in said unit dose when taken together is more effective to treat the subject than when compared to the administration of said composition in the absence of said opioid or opiate or the administration of said opioid or opiate in the absence of said composition.

A pharmaceutical composition in unit dosage form, useful in treating a subject afflicted with pain, a depressive disorder, a mood disorder, an anxiety disorder, a substance use disorder, opioid withdrawal symptoms, traumatic brain injury or Parkinson’s disease, which comprises:
(i) a composition comprising the compound of the present invention; and
(ii) an amount of an opioid or opiate,
   wherein the respective amounts of said composition and said opioid or opiate in said composition are effective, upon concomitant administration to said subject of one or more of said unit dosage forms of said composition, to treat the subject.

The pharmaceutical composition of the above embodiment, wherein the respective amounts of said compound and said opioid or opiate in said unit dose when taken together is more effective to treat the subject than when compared to the administration of said composition in the absence of said opioid or opiate or the administration of said opioid or opiate in the absence of said composition.

In some embodiments of the present method, package, use or pharmaceutical composition, the compound has the structure:

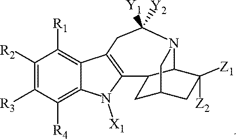

In some embodiments, a pharmaceutically acceptable salt of any of the above compounds of the present invention.

In some embodiments, a salt of the compound of the present invention is used in any of the above methods, uses, packages or compositions.

In some embodiments, a pharmaceutically acceptable salt of the compound of the present invention is used in any of the above methods, uses, packages or compositions.

Any of the above compounds may be used in any of the disclosed methods, uses, packages or pharmaceutical compositions.

Any of the compounds used in the disclosed methods, uses, packages or pharmaceutical compositions may be replaced with any other compound disclosed in the present invention.

Any of the above generic compounds may be used in any of the disclosed methods, uses, packages or compositions.

In some embodiments of any of the above methods, wherein the composition is orally administered to the subject.

In some embodiments of any of the above methods, wherein 10 - 30 mg of the compound is administered to the subject.

In some embodiments of any of the above methods, wherein 30 - 100 mg of the compound is administered to the subject.

In some embodiments of any of the above methods, wherein 100 - 300 mg of the compound is administered to the subject.

In some embodiments of any of the above methods, wherein 300 - 500 mg of the compound is administered to the subject.

In some embodiments of any of the above methods, wherein 500 - 800 mg of the compound is administered to the subject.

In some embodiments of any of the above methods, wherein 800 - 1100 mg of the compound is administered to the subject.

In some embodiments of any of the above methods, wherein 1200 - 1500 mg of the compound is administered to the subject.

In some embodiments, a method wherein any of the above recited doses of the compound, and an opioid are administered to a subject afflicted with a substance use disorder, opioid withdrawal symptoms, pain, a mood disorder, an anxiety disorder or opioid cravings so as to thereby treat the subject afflicted with the substance use disorder, opioid withdrawal symptoms, pain or the mood disorder or reduce opioid cravings in the subject.

In some embodiments of any of the above methods, wherein the opioid is morphine and 10-20 mg (oral) or 3-5 mg (parenteral) of the opioid is administered to the subject.

In some embodiments of any of the above methods, wherein the opioid is codeine and 30-60 mg (oral) of the opioid is administered to the subject.

In some embodiments of any of the above methods, wherein the opioid is oxycodone and 5-10 mg (oral) of the opioid is administered to the subject.

In some embodiments of any of the above methods, wherein the opioid is fentanyl and 40-60 µg (parenteral) of the opioid is administered to the subject.

In some embodiments of any of the above methods, wherein the opioid is butorphanol and 1-3 mg (parenteral) of the opioid is administered to the subject.

In some embodiments of any of the above methods, wherein the opioid is nalbuphine and 5-15 mg (parenteral) of the opioid is administered to the subject.

In some embodiments of any of the above methods, wherein mitragynine (15-100 mg - oral) or 3-deuteromitragynine (15-100 mg - oral) is administered to the subject.

In some embodiments of any of the above methods, wherein tianeptine (12.5-100 mg - oral) is administered to the subject.

In some embodiments of any of the above methods, wherein 7-((3-iodo-6-methyl-5,5-dioxido-6,11-dihydrodibenzo[c,f][1,2]thiazepine-11-yl)amino)heptanoic acid (1.5-10 mg - oral) is administered to the subject.

In some embodiments of any of the above methods, wherein 5-((3-iodo-6-methyl-5,5-dioxido-6,11-dihydrodibenzo[c,f][1,2]thiazepine-11-yl)amino)pentanoic acid (2-20 mg - oral) is administered to the subject.

In some embodiments of any of the above methods, wherein administration of the composition of the present invention comprising an effective amount of the compound lowers the effective amount of the opioid.

In some embodiments of any of the above methods, wherein administration of the composition of the present invention lowers the effective dosage amount of the opioid by 75% or more.

In some embodiments of the above method, wherein administration of the composition of the present invention lowers the effective dosage amount of the opioid by 50% or more.

In some embodiments of the above method, wherein administration of the composition of the present invention lowers the effective dosage amount of the opioid by 25% or more.

In some embodiments of any of the above methods, wherein 0.4 mg/kg -30 mg/kg of the compound of the present invention is administered to the subject.

In some embodiments of any of the above methods, wherein 0.3-1.5 mg/kg of the opioid or opiate is administered to the subject.

In some embodiments of any of the above methods, wherein the subject is a human.

In some embodiments of any of the above methods, the composition is clinic administered or physician administered to the subject.

In some embodiments of any of the above methods, the composition is clinic self-administered by the subject.

In some embodiments, of any of the above methods, wherein formation of noribogaine is attenuated within the subject.

In some embodiments of any of the above methods, wherein formation of noribogaine is reduced within the subject.

In some embodiments of any of the above methods, wherein metabolism of ibogaine is attenuated within the subject.

In some embodiments of any of the above methods, wherein metabolism of ibogaine is enhanced within the subject.

In some embodiments of any of the above methods, wherein metabolism of 10-ethoxy-ibogamine is attenuated within the subject.

In some embodiments of any of the above methods, wherein metabolism of 10-ethoxy-ibogamine is enhanced within the subject.

In some embodiments, the method wherein the subject is afflicted with a depressive disorder, a mood disorder, or an anxiety disorder.

In some embodiments, the anxiety disorder includes, but is not limited to, anxiety, generalized anxiety disorder (GAD), panic disorder, social phobia, social anxiety disorder, acute stress disorder, obsessive-compulsive disorder (OCD), or post-traumatic stress disorder (PTSD).

In some embodiments, the depressive disorder includes, but is not limited to, depression, major depression, dysthymia, cyclothymia, postpartum depression, seasonal affective disorder, atypical depression, psychotic depression, bipolar disorder, premenstrual dysphoric disorder, situational depression or adjustment disorder with depressed mood. Depressive disorders can also include other mood disorders and is not limited to the above list.

Preclinical evidence (rodents) also shows that ibogaine/noribogaine enhances morphine’s analgesic effect (Sharma, S.S. et al. 1998) or reverses analgesic tolerance to morphine (Bhargava, H.N. et al. 1997).

In some embodiments, the method wherein the subject is afflicted with pain. Reports of stimulant effects of Tabernanthe iboga date back to late 1890′s and early 1900′s in the descriptions of ritual and medicinal use by the native inhabitants in Africa. Ibogaine was recommended in France to treat “asthenia” (dose range of 10-30 mg per day). In the period of 1939-1970, ibogaine was commercially available in France as “Lambarène”, a “neuromuscular stimulant” (8 mg pills) recommended for fatigue, depression, and recovery from infectious diseases (Alper, K.R. 2001). In one clinical study, subjects took visual analog scale tests (VAS, 0-100) related to sleepiness, energetic feelings, and the side effects such as nausea, anxiety versus calmness. Subjects reported that ibogaine decreased sleepiness and increased energetic feeling over the examined 24-hour period after one dose of 20 mg of ibogaine (Glue, P. et al. 2015). A stimulant effect was reported in cats (Schneider et. Al 1957). In rats, ibogaine induced wakefulness and suppressed the REM sleep as shown via EEG (González, J. et al 2018).

It has been shown in rats that ibogaine leads to a dramatic upregulation of BDNF (in addition to Glial cell line-Derived Neurotrophic Factor (GDNF)) which provides structural and functional restorative effects in subjects afflicted with TBI (Marton, S. et al. 2019). The efficacy of ibogaine has also been shown in cases of soldiers afflicted with TBI and PTSD (Thoricatha, W. 2020).

In some embodiments, the method wherein the subject is afflicted with traumatic brain injury (TBI).

It has been shown in rats that ibogaine induces expression of GDNF (He, D-Y. et al. 2005 and Marton, S. et al. 2019), a critical neurotrophic factor that maintains and restores the dopaminergic system (which degenerates in Parkinson’s disease). Thus, ibogaine provides structural and functional restorative effects in subjects afflicted with Parkinson’s disease. GDNF itself has been shown to exert desired effects in Parkinson’s rodent and monkey models (Gash, D.M. et al. 1996).

In some embodiments, the method wherein the subject is afflicted with Parkinson’s disease.

It has been shown in humans that ibogaine is useful in treating opioid and stimulant use disorders (Alper, K.R. et al. 1999; Mash, D.C. et al. 2018; Schenberg, E.E. et al. 2014) or in maintenance therapy (opioid use disorder) in combination with an opioid to lower effective opioid doses (Kroupa, P.K. & Wells, H. 2005).

In some embodiments, wherein the substance use disorder is an opioid use disorder, alcohol use disorder or stimulant use disorder.

Opioid use disorder (OUD) involves, but is not limited to, misuse of opioid medications or use of illicitly obtained opioids. The Diagnostic and Statistical Manual of Mental Disorders, 5^{th} Edition (American Psychiatric Association: Diagnostic and Statistical Manual of Mental Disorders: Diagnostic and Statistical Manual of Mental Disorders, Fifth Edition. Arlington, VA:

American Psychiatric Association, 2013), which is hereby incorporated by reference, describes opioid use disorder as a problematic pattern of opioid use leading to problems or distress, with at least two of the following occurring within a 12-month period:
-Taking larger amounts or taking drugs over a longer period than intended.
-Persistent desire or unsuccessful efforts to cut down or control opioid use.
-Spending a great deal of time obtaining or using the opioid or recovering from its effects.
-Craving, or a strong desire or urge to use opioids.
-Problems fulfilling obligations at work, school, or home.
-Continued opioid use despite having recurring social or interpersonal problems.
-Giving up or reducing activities because of opioid use.
-Using opioids in physically hazardous situations.
-Continued opioid use despite ongoing physical or psychological problem likely to have been caused or worsened by opioids.
-Tolerance (i.e., need for increased amounts or diminished effect with continued use of the same amount).
-Experiencing withdrawal (opioid withdrawal syndrome) or taking opioids (or a closely related substance) to relieve or avoid withdrawal symptoms.

Alcohol use disorder (AUD) involves, but is not limited to, a chronic relapsing brain disease characterized by compulsive alcohol use, loss of control over alcohol intake, and a negative emotional state when not using. The Diagnostic and Statistical Manual of Mental Disorders, 5^{th} Edition describes alcohol use disorder as a problematic pattern of alcohol use leading to problems or distress, with at least two of the following occurring within a 12-month period:
-Being unable to limit the amount of alcohol you drink.
-Wanting to cut down on how much you drink or making unsuccessful attempts to do so.
-Spending a lot of time drinking, getting alcohol, or recovering from alcohol use.
-Feeling a strong craving or urge to drink alcohol.
-Failing to fulfill major obligations at work, school or home due to repeated alcohol use.
-Continuing to drink alcohol even though you know it is causing physical, social, or interpersonal problems.
-Giving up or reducing social and work activities and hobbies.
-Using alcohol in situations where it is not safe, such as when driving or swimming.
-Developing a tolerance to alcohol so you need more to feel its effect, or you have a reduced effect from the same amount.
-Experiencing withdrawal symptoms — such as nausea, sweating and shaking — when you do not drink, or drinking to avoid these symptoms.

Stimulant use disorder involves, but is not limited to, a pattern of problematic use of amphetamine, methamphetamine, cocaine, or other stimulants except caffeine or nicotine, leading to at least two of the following problems within a 12-month period:
-Taking more stimulants than intended.
-Unsuccessful in trying to cut down or control use of stimulants, despite wanting to do so.
-Spending excessive amounts of time to activities surrounding stimulant use.
-Urges and cravings for stimulants.
-Failing in the obligations of home, school, or work.
-Carrying on taking stimulants, even though it has led to relationship or social problems.
-Giving up or reducing important recreational, social, or work-related activities because of using stimulants.
-Using stimulants in a physically hazardous way.
-Continuing to use stimulants even while knowing that it is causing or worsening a physical or psychological problem.
-Tolerance to stimulants.
-Withdrawal from stimulants if you do not take them.

Polydrug use disorder or polysubstance use disorder involves, but is not limited to, dependence on multiple drugs or substances.

The term “MOR agonist” is intended to mean any compound or substance that activates the mu-opioid receptor (MOR). The agonist may be a partial, full, or super agonist.

A person skilled in the art may use the techniques disclosed herein to prepare deuterium analogs thereof.

Except where otherwise specified, the structure of a compound of this invention includes an asymmetric carbon atom, it is understood that the compound occurs as a racemate, racemic mixture, scalemic mixtures and isolated single enantiomers. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

Except where otherwise specified, the subject invention is intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notations of a carbon in structures throughout this application, when used without further notation, are intended to represent all isotopes of carbon, such as ¹²C, ¹³C, or ¹⁴C.

Furthermore, any compounds containing ¹³C or ¹⁴C may specifically have the structure of any of the compounds disclosed herein.

It will also be noted that any notations of a hydrogen (H) in structures throughout this application, when used without further notation, are intended to represent all isotopes of hydrogen, such as ¹H, ²H (D), or ³H (T) except where otherwise specified. Furthermore, any compounds containing ²H (D) or ³H (T) may specifically have the structure of any of the compounds disclosed herein except where otherwise specified.

Isotopically labeled compounds can generally be prepared by conventional techniques known to those skilled in the art using appropriate isotopically labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (²H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart.

As used herein, a hydrogen at a specific site in a compound is “deuterium-enriched” if the amount of deuterium at the specific site in the compound is more than the abundance of deuterium naturally occurring at that specific site in view of all of the molecules of the compound in a defined universe such as a composition or sample. Naturally occurring as used above refers to the abundance of deuterium which would be present at a relevant site in a compound if the compound was prepared without any affirmative step to enrich the abundance of deuterium. Thus, at a “deuterium-enriched” site in a compound, the abundance of deuterium at that site can range from more than 0.0156% to 100%. Examples of ways to obtain a deuterium-enriched site in a compound are exchanging hydrogen with deuterium or synthesizing the compound with deuterium-enriched starting materials.

In the compounds used in the method of the present invention, the substituents may be substituted or unsubstituted, unless specifically defined otherwise.

In the compounds used in the method of the present invention, alkyl, alkenyl, alkynyl, alkylaryl, cycloalkyl, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2......, n-1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge. The term “alkenyl” refers to a non-aromatic hydrocarbon radical, straight or branched, containing at least 1 carbon to carbon-to-carbon double bond, and up to the maximum possible number of non-aromatic carbon-carbon double bonds may be present. Thus, C₂-Cₙ alkenyl is defined to include groups having 1, 2...., n-1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched, or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon-to-carbon triple bond, and up to the maximum possible number of non-aromatic carbon-carbon triple bonds may be present. Thus, C₂-Cₙ alkynyl is defined to include groups having 1, 2...., n-1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₈ alkynyl.

The term “alkylaryl” refers to alkyl groups as described above wherein one or more bonds to hydrogen contained therein are replaced by a bond to an aryl group as described above. It is understood that an “alkylaryl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

As used herein, “cycloalkyl” includes cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic, or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, 53hiazepine53e, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, 53hiazepin, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R-O-CO-R′ group.

The term “phenyl” is intended to mean an aromatic six membered ring containing six carbons.

The term “benzyl” is intended to mean a -CH₂R₁ group wherein the R₁ is a phenyl group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, C1, Br, and I); alkyl groups, such as methyl, ethyl, n-propyl, isopropyl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The term “soft nucleophile” is intended to mean a nucleophile with high polarizability and low electronegativity.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel’s Textbook of Practical Organic Chemistry, A.I. Vogel, A.R. Tatchell, B.S. Furnis, A.J. Hannaford, P.W.G. Smith, (Prentice Hall) 5^{th} Edition (1996), March’s Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Another aspect of the invention comprises a compound or composition of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians’ Desk Reference (PDR Network, LLC; 64^{th} edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent’s biological activity or effect.

The compounds used in the method of the present invention may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect, refers to the relatively non-toxic, inorganic, and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease. Treating may also mean improving one or more symptoms of a disease.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier, as are capsules, coatings, and various syringes.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous, or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin, and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavoring and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2^{nd} Edition (1976); Remington’s Pharmaceutical Sciences, 17^{th} ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington’s Pharmaceutical Sciences, 17^{th} ed., 1989, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention. Any of the disclosed generic or specific compounds may be applicable to any of the disclosed compositions, processes, or methods.

This invention will be better understood by reference to the Experimental Details which follow, but those skilled in the art will readily appreciate that the specific experiments detailed are only illustrative of the invention as described more fully in the claims, which follow thereafter.

### EXPERIMENTAL DETAILS

General Considerations. Reagents and solvents were obtained from commercial sources and were used without further purification unless otherwise stated. Reactions were monitored by TLC using solvent mixtures appropriate to each reaction. Column chromatography was performed mainly on silica gel (40 - 63 µm) or basic alumina (50 -200 µm) as indicated. For compounds containing a basic nitrogen, Et₃N was often used in the mobile phase to provide better resolution when using silica gel chromatography. In these cases, TLC plates were pre-soaked in the Et₃N-containing solvent and then allowed to dry briefly before use in analysis, such that an accurate representation of R_{f} was obtained. For PTLC, glass plates coated with a 1 mm silica layer were used. Nuclear magnetic resonance spectra were recorded on Bruker 400 or 500 MHz instruments, as indicated. Chemical shifts are reported as δ values in ppm referenced to CDCl₃ (¹H NMR = 7.26 and ¹³C NMR = 77.16) or methanol-d₄ (¹H NMR = 3.31 and ¹³C NMR = 49.00). Multiplicity is indicated as follows: s (singlet); d (doublet); t (triplet); dd (doublet of doublets); td (triplet of doublets); dt (doublet of triplets); dq (doublet of quartets); ddd (doublet of doublet of doublets); ddt (doublet of doublet of triplets); m (multiplet); br (broad). All carbon peaks are rounded to one decimal place unless such rounding would cause two close peaks to become identical; in these cases, two decimal places are retained. Low-resolution mass spectra were recorded on an Advion quadrupole instrument (ionization mode: APCI+).

Synthetic Procedures and Characterizations of Iboga Analogs Noribogaine were prepared as previously described (Rodriguez, P. et al. 2020).

### Example 1. Synthesis of Ibogamine-10-triflate 1

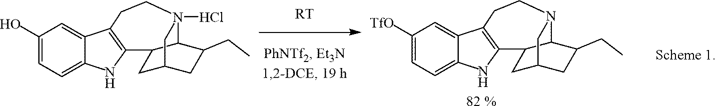

### Ibogamine-10-triflate 1

Noribogaine hydrochloride (100 mg, 0.3 mmol) and bis(trifluoromethanesulfonyl)aniline (118 mg, 0.33 mmol) were suspended in 1,2-dichloroethane (anhydrous, 2.2 mL) and Et₃N (125 µL, 0.9 mmol) was added. After stirring at RT for 19 h, reaction was quenched by pouring into H₂O (15 mL) and the mixture was extracted with CH₂Cl₂ (3 × 10 mL). Combined extracts were dried over Na₂SO₄, filtered, and concentrated under reduced pressure. Crude material was purified by column chromatography (gradient of 10 to 20% AcOEt in hexanes + 2% Et₃N). 1 was obtained as an off-white amorphous solid (106 mg, 82%).

¹H NMR (500 MHz, CDCl₃) δ 7.79 (s, 1H), 7.33 (d, J = 2.4 Hz, 1H), 7.24 (d, J = 8.7 Hz, 1H), 6.98 (dd, J = 8.7, 2.4 Hz, 1H), 3.41 - 3.31 (m, 2H), 3.21 - 3.09 (m, 1H), 3.06 (dt, J = 9.4, 2.3 Hz, 1H), 2.99 (dt, J = 9.4, 3.0 Hz, 1H), 2.96 - 2.90 (m, 1H), 2.86 (t, J = 1.7 Hz, 1H), 2.64 - 2.53 (m, 1H), 2.14 - 2.00 (m, 1H), 1.90 - 1.84 (m, 1H), 1.84 -1.77 (m, 1H), 1.69 - 1.61 (m, 1H), 1.61 - 1.50 (m, 2H), 1.50 - 1.42 (m, 1H), 1.25 - 1.18 (m, 1H), 0.90 (t, J = 7.1 Hz, 3H). ¹⁹F NMR (471 MHz, CDCl₃) δ -71.9. ¹³C NMR (126 MHz, CDCl₃) δ 144.9, 143.6, 133.5, 130.3, 122.8, 120.3, 117.7, 115.2, 113.9, 111.0, 110.6, 110.4, 57.5, 54.1, 50.1, 42.1, 41.7, 34.2, 32.1, 28.0, 26.6, 20.7, 12.0. LRHS (APCI+) calcd. For C₂₀H₂₄F₃N₂O₃S⁺ [M+H]⁺ 429.1, found 429.0.

### Example 2. Synthesis of Ibogamine-10-carboxamide 2

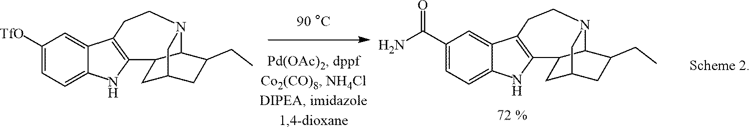

### Ibogamine-10-carboxamide 2

Compound 1 (70 mg, 0.16 mmol), Pd(Oac)₂ (1.8 mg, 0.008 mmol), dppf (9.0 mg, 0.016 mmol), Co₂(CO)₈ (33 mg, 0.097 mmol), imidazole (2.8 mg, 0.041 mmol) and NH₄Cl (35 mg, 0.65 mmol) were balanced into a reaction vial. Under stream of argon, 1,4-dioxane (anhydrous, 3.3 mL) and DIPEA (114 µL, 0.65 mmol) were added, vial was closed with a Teflon lined solid screw cap and the RM was heated to 90° C. After 14 h, the RM was cooled to RT and purified by column chromatography (gradient of 5, 10 and 15% MeOH in a 1:1 mixture of AcOEt and hexanes). Material was further purified by using PTLC (5% MeOH in CH₂Cl₂ + 2% Et₃N), coeluting Et₃N salts were removed by washing a CH₂Cl₂ solution of the product with 2 M aq. Na₂CO₃ solution. 2 was isolated as yellow solid (38 mg, 72%).

¹H NMR (500 MHz, CDCl₃ + MeOD) δ 7.86 (d, J = 1.8 Hz, 1H), 7.42 (dd, J = 8.4, 1.8 Hz, 1H), 7.12 (d, J = 8.5 Hz, 1H), 3.23 - 3.15 (m, 2H), 3.00 - 2.92 (m, 1H), 2.90 - 2.81 (m, 2H), 2.81 - 2.74 (m, 1H), 2.68 (s, 1H), 2.64 - 2.52 (m, 1H), 1.97 - 1.88 (m, 1H), 1.73 - 1.63 (m, 2H), 1.47 (dq, J = 13.4, 3.5 Hz, 1H), 1.44 - 1.35 (m, 2H), 1.35 - 1.27 (m, 1H), 1.08 - 1.02 (m, 1H), 0.75 (t, J = 7.2 Hz, 3H). ¹³C NMR (126 MHz, CDCl₃ + MeOD) δ 172.4, 143.6, 137.1, 129.0, 123.0, 119.9, 118.0, 109.9, 109.3, 57.6, 53.9, 49.6, 41.8, 40.8, 33.7, 31.7, 27.4, 26.2, 20.3, 11.6. LRMS (APCI+) calcd. For C₂₀H₂₆N₃O⁺ [M+H]⁺ 324.2, found 324.2.

### Example 3. Synthesis of Ibogamine-10-carbonitrile 3

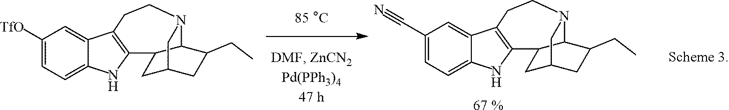

### Ibogamine-10-carbonitrile 3

Compound 1 (43 mg, 0.1 mmol), ZnCN₂ (24 mg, 0.2 mmol), Pd (PPh₃) ₄ (5.8 mg, 0.005 mmol) were balanced into a reaction vial and dried under vacuum. DMF (anhydrous, 0.5 mL) was added, vial was flushed with argon, closed with a Teflon lined solid screw cap and the RM heated to 85° C. After 24 h, TLC indicated only partial conversion. Additional ZnCN₂ (47 mg, 0.4 mmol) and Pd(PPh₃)₄ (12 mg, 0.01 mmol) were added and heating was continued for additional 23 h, until TLC indicated that no more SM remained. RM was diluted with H₂O (10 mL) and sat. aq. NaHCO₃ solution (2 mL). Mixture was extracted with Et₂O (3 × 5 mL) and AcOEt (3 × 5 mL). Extraction is complicated by the presence of insoluble solid precipitates. Combined extracts were washed with H₂O, brine, dried over Na₂SO₄, filtered and concentrated. Crude material was purified by column chromatography (gradient of 10, 20 and 25% of AcOEt in hexanes + 2% Et₃N). 3 was isolated as a white amorphous solid (21 mg, 67%).

¹H NMR (500 MHz, CDCl₃) δ 8.15 (s, 1H), 7.79 (s, 1H), 7.33 (d, J = 8.2 Hz, 1H), 7.29 (d, J = 8.3 Hz, 1H), 3.43 - 3.30 (m, 2H), 3.19 - 3.09 (m, 1H), 3.09 - 2.93 (m, 3H), 2.85 (s, 1H), 2.66 - 2.57 (m, 1H), 2.13 - 2.02 (m, 1H), 1.90 - 1.84 (m, 1H), 1.84 - 1.76 (m, 1H), 1.64 (dq, J = 13.9, 3.4 Hz, 1H), 1.60 - 1.50 (m, 2H), 1.50 - 1.41 (m, 1H), 1.25 -1.16 (m, 1H), 0.89 (t, J = 7.0 Hz, 3H). ¹³C NMR (126 MHz, CDCl₃) δ 144.4, 136.6, 129.8, 124.1, 123.5, 121.3, 111.0, 110.3, 102.0, 57.5, 54.0, 50.1, 42.0, 41.5, 34.2, 32.1, 27.9, 26.5, 20.6, 12.0. LRMS (APCI+) calcd. For C₂₀H₂₄N₃⁺ [M+H]⁺ 306.2, found 306.1.

### Example 4. Synthesis of 10-Propoxy-ibogamine 4

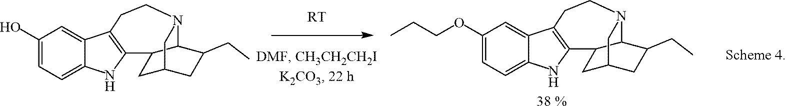

### 10-Propoxy-ibogamine 4

Noribogaine (63 mg, 0.19 mmol) and K₂CO₃ (79 mg, 0.57 mmol) were mixed in DMF (anhydrous, 0.5 mL). CH₃CH₂CH₂I (37 µL, 0.38 mmol) was added into the suspension and RM was stirred at RT for 22 h. Reaction was quenched by adding H₂O (15 mL) and the mixture was extracted with Et₂O (3 × 5 mL) and toluene (5 mL). Combined org. extracts were washed with brine (2 mL), dried over Na₂SO₄, filtered, and concentrated under reduced pressure. Crude material was purified by column chromatography (20% AcOEt in hexanes, to 20 % AcOEt in hexanes + 2% Et₃N) followed by PTLC (10% Et₂O in hexanes + 2 % Et₃N). 4 was isolated as a pale-yellow amorphous solid (24 mg, 38%).

¹H NMR (500 MHz, CDCl₃) δ 7.52 (s, 1H), 7.13 (d, J = 8.6 Hz, 1H), 6.95 (d, J = 2.4 Hz, 1H), 6.78 (dd, J = 8.6, 2.4 Hz, 1H), 3.98 (t, J = 6.7 Hz, 2H), 3.42 - 3.29 (m, 2H), 3.18 - 3.10 (m, 1H), 3.08 (dt, J = 9.3, 2.3 Hz, 1H), 2.98 (dt, J = 9.3, 3.0 Hz, 1H), 2.90 - 2.83 (m, 2H), 2.64 - 2.57 (m, 1H), 2.03 (ddt, J = 13.2, 11.6, 2.7 Hz, 1H), 1.87 - 1.77 (m, 4H), 1.68 - 1.62 (m, 1H), 1.60 - 1.51 (m, 2H), 1.51 - 1.44 (m, 1H), 1.24 - 1.18 (m, 1H), 1.06 (t, J = 7.4 Hz, 3H), 0.90 (t, J = 7.2 Hz, 3H). ¹³C NMR (126 MHz, CDCl₃) δ 153.5, 143.0, 130.3, 129.9, 111.4, 110.8, 109.2, 101.8, 70.8, 57.6, 54.3, 50.1, 42.1, 41.7, 34.3, 32.3, 28.0, 26.7, 23.0, 20.9, 12.1, 10.8. LRMS (APCI+) calcd. For C₂₂H₃₁N₂O⁺ [M+H]⁺ 339.2, found 339.3.

### Example 5. Synthesis of Ibogamine 5

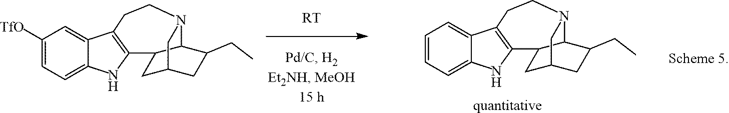

### Ibogamine 5

Compound 1 (20 mg, 0.047 mmol) was dissolved in MeOH (not dry, 1.4 mL) Pd/C (10%, moistened, 2 mg) was added and the RM was purged with H₂ (sparging using a needle and balloon). Diethylamine (6 µL, 0.056 mmol) was added and the RM was further stirred under H₂ atmosphere (1 atm., balloon). After 4 h, TLC indicated that most SM was consumed. After 15 h, the RM was filtered through a filter paper and the solids rinsed with MeOH. Filtrate was concentrated, re-dissolved in CH₂Cl₂ and washed with 2 M aq. Na₂CO₃ solution. The CH₂Cl₂ solution was dried over Na₂SO₄, filtered, and concentrated to yield 5 as a pale yellow amorphous solid (13 mg, quantitative).

¹H NMR (500 MHz, CDCl₃) δ 7.63 (s, 1H), 7.48 (dd, J = 7.2, 1.6 Hz, 1H), 7.28 - 7.24 (m, 1H), 7.15 - 7.07 (m, 2H), 3.42 - 3.33 (m, 2H), 3.20 -3.11 (m, 1H), 3.08 (dt, J = 9.3, 2.3 Hz, 1H), 2.98 (dt, J = 9.3, 3.0 Hz, 1H), 2.96 - 2.90 (m, 1H), 2.89 - 2.85 (m, 1H), 2.72 - 2.66 (m, 1H), 2.09 - 2.01 (m, 1H), 1.88 - 1.83 (m, 1H), 1.83 - 1.78 (m, 1H), 1.69 - 1.63 (m, 1H), 1.59 - 1.53 (m, 2H), 1.51 - 1.43 (m, 1H), 1.26 -1.19 (m, 1H), 0.91 (t, J = 7.1 Hz, 3H). ¹³C NMR (126 MHz, CDCl₃) δ 141.97, 134.77, 129.86, 121.08, 119.23, 118.04, 110.21, 109.37, 57.71, 54.30, 50.07, 42.11, 41.63, 34.34, 32.27, 27.95, 26.66, 20.80, 12.07. LRMS (APCI+) calcd. For C₁₉H₂₅N₂⁺ [M+H]⁺ 281.2, found 281.5.

### Example 6. Synthesis of N-methyl-ibogamine 6

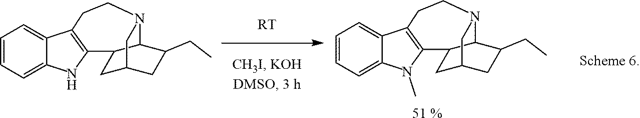

### N-methyl-ibogramine 6

Ibogamine 5 (50 mg, 0.18 mmol) and KOH (20 mg, 0.36 mmol) were mixed in DMSO (anhydrous, 0.3 mL). CH₃I (17 µL, 0.27 mmol) was added into the suspension and RM was stirred at RT for 3 h. Reaction was quenched by adding H₂O (15 mL) and the mixture was extracted with CH₂Cl₂ (3 × 5 mL). Combined extracts were dried over Na₂SO₄, filtered, and concentrated under reduced pressure. Crude material was purified by PTLC (1:60 AcOEt in hexanes + 2% Et₃N, developed twice). 6 was isolated as a yellow waxy solid (27 mg, 51%).

¹H NMR (500 MHz, CDCl₃) δ 7.52 (d, J = 7.8 Hz, 1H), 7.30 - 7.25 (m, 1H), 7.24 - 7.17 (m, 1H), 7.15 - 7.07 (m, 1H), 3.68 (s, 3H), 3.48 -3.34 (m, 2H), 3.27 - 3.20 (m, 1H), 3.20 - 3.14 (m, 1H), 3.12 - 3.02 (m, 2H), 2.93 (s, 1H), 2.85 - 2.78 (m, 1H), 2.16 - 2.07 (m, 1H), 1.94 - 1.84 (m, 2H), 1.71 - 1.46 (m, 4H), 1.38 - 1.24 (m, 1H), 0.97 (t, J = 7.2 Hz, 3H). ¹³C NMR (126 MHz, CDCl₃) δ 143.2, 136.2, 128.3, 120.8, 118.8, 118.1, 108.9, 108.8, 57.5, 54.4, 50.4, 42.5, 38.6, 33.7, 32.2, 29.8, 27.9, 26.6, 21.1, 12.1. LRMS (APCI+) calcd. For C₂₀H₂₇N₂⁺ [M+H]⁺ 295.2, found 295.7.

### Example 7. Synthesis of N-methyl-ibogaine 7

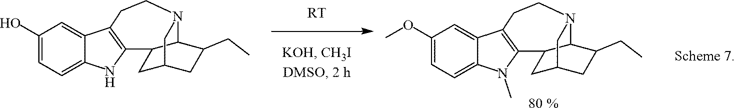

### N-methyl-ibogaine 7

Noribogaine (200 mg, 0.67 mmol) and KOH (150 mg, 2.7 mmol) were mixed in DMSO (anhydrous, 2.0 mL). After 1 h, CH₃I (125 µL, 2.0 mmol) was added and the stirring continued. Reaction mixture was poured into H₂O after 1 h and extracted with Et₂O (3 × 15 mL). Combined extracts were washed with H₂O and brine, dried over Na₂SO₄, filtered, and concentrated under reduced pressure. Crude material was purified by column chromatography (1:20, AcOEt in hexanes + 2% Et₃N). 7 was isolated as a pale-yellow viscous oil (174 mg, 80%).

¹H NMR (500 MHz, CDCl₃) δ 7.11 (d, J = 8.7 Hz, 1H), 6.94 (d, J = 2.4 Hz, 1H), 6.82 (dd, J= 8.7, 2.4 Hz, 1H), 3.86 (s, 3H), 3.61 (s, 3H), 3.39 - 3.28 (m, 2H), 3.23 - 3.12 (m, 1H), 3.12 - 3.02 (m, 2H), 2.98 (dt, J = 9.4, 2.9 Hz, 1H), 2.85 (t, J = 1.6 Hz, 1H), 2.76 - 2.64 (m, 1H), 2.11 - 1.99 (m, 1H), 1.91 - 1.79 (m, 2H), 1.65 - 1.52 (m, 3H), 1.52 - 1.42 (m, 1H), 1.29 - 1.19 (m, 1H), 0.91 (t, J = 7.2 Hz, 3H). ¹³C NMR (126 MHz, CDCl₃) δ 153.9, 144.1, 131.6, 128.5, 110.6, 109.5, 108.6, 100.5, 57.5, 56.3, 54.4, 50.5, 42.5, 38.8, 33.8, 32.2, 29.9, 28.0, 26.6, 21.2, 12.1. LRMS (APCI+) calcd. For C₂₁H₂₉N₂O⁺ [M+H]⁺ 325.2, found 325.2.

### Example 8. Synthesis of N-methyl-noribogaine 8

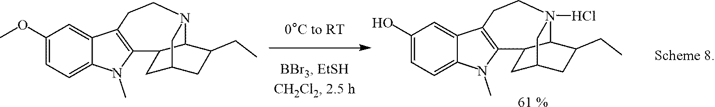

### N-methyl-noribogaine 8

N-Methyl-ibogaine 7 (80 mg, 0.25 mmol) was dissolved in CH₂Cl₂ (anhydrous, 1.6 mL) and cooled in ice bath. EtSH (0.079 mL, 1.1 mmol) and BBr₃ (1 M in CH₂Cl₂, 0.37 mL) were added and the RM was further stirred at RT for 1.5 h - significant amount of SM remained. Additional EtSH (0.027 mL, 1.1 mmol) and BBr₃ (1 M in CH₂Cl₂, 0.12 mL) were added, reaction was stirred for 1 h more and quenched by pouring into sat. aq. NaHCO₃ solution (20 mL). The mixture was extracted with CH₂Cl₂ (3 × 10 mL), combined extracts were dried over Na₂SO₄, filtered, and concentrated under reduced pressure. Crude material was pre-purified by column chromatography (gradient of 1:4 to 1:2 AcOEt in hexanes + 2 % Et₃N), isolated fraction was evaporated from a mixture of MeOH/H₂O to remove excess Et₃N. The free base was further dissolved in MeOH, acidified with HCl (36% aq.) to pH 1-2, evaporated to dryness and washed with CH₃CN (3 × 1 mL, solid was sedimented by centrifugation and solvent decanted). 8 was isolated as a pale yellow amorphous solid (86 mg, 61%) as a hydrochloride salt.

¹H NMR (500 MHz, MeOD) δ 7.15 (d, J = 8.7 Hz, 1H), 6.84 (d, J = 2.3 Hz, 1H), 6.72 (dd, J = 8.7, 2.3 Hz, 1H), 3.74 - 3.67 (m, 2H), 3.67 -3.60 (m, 4H), 3.59 - 3.53 (m, 1H), 3.50 - 3.37 (m, 2H), 3.28 - 3.11 (m, 2H), 2.44 - 2.36 (m, 1H), 2.24 - 2.19 (m, 1H), 2.19 - 2.08 (m, 2H), 1.75 - 1.58 (m, 3H), 1.47 - 1.40 (m, 1H), 1.07 (t, J = 7.3 Hz, 3H). ¹³C NMR (126 MHz, MeOD) δ 152.0, 141.2, 132.9, 129.1, 112.6, 110.7, 107.6, 103.1, 60.8, 58.4, 53.1, 40.3, 34.2, 31.8, 29.9, 29.9, 27.5, 25.4, 19.8, 12.0. LRMS (APCI+) calcd. For C₂₀H₂₇N₂O⁺ [M+H]⁺ 311.2, found 311.7.

### Example 9. Synthesis of N-methyl-ibogamine-10-triflate 9

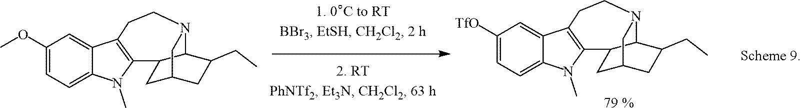

### N-methyl-ibogramine-10-triflate 9

N-Methyl-ibogaine 7 (312 mg, 0.96 mmol) was dissolved in CH₂Cl₂ (anhydrous, 6.5 mL) and cooled in ice bath. EtSH (0.32 mL, 4.4 mmol) and BBr₃ (1 M in CH₂Cl₂, 1.4 mL) were added, RM was further stirred at RT for 2 h and quenched by pouring into sat. aq. NaHCO₃ solution (30 mL). The mixture was extracted with CH₂Cl₂ (3 × 10 mL), combined extracts were dried over Na₂SO₄, filtered, and concentrated under reduced pressure. Crude material was dried on high vacuum for 4 h and used for the next step without further purification.

The crude N-Me-noribogaine and bis(trifluoromethanesulfonyl)aniline (343 mg, 0.96 mmol) were dissolved in CH₂Cl₂ (anhydrous, 5 mL) and Et₃N (0.2 mL, 1.4 mmol) was added. After stirring at RT for 63 h, reaction was quenched by pouring into 2 M aq. Na₂CO₃ solution (20 mL) and the mixture was extracted with CH₂Cl₂ (3 × 10 mL). Combined extracts were dried over Na₂SO₄, filtered, and concentrated under reduced pressure. Crude material was purified by column chromatography (basic alumina, hexanes, and hexanes + 2% AcOEt). 9 was isolated as a pale-yellow viscous oil (334 mg, 79% over two steps).

¹H NMR (500 MHz, CDCl₃) δ 7.33 (d, J = 2.4 Hz, 1H), 7.20 (d, J = 8.8 Hz, 1H), 7.03 (dd, J = 8.8, 2.5 Hz, 1H), 3.65 (s, 3H), 3.39 - 3.30 (m, 2H), 3.22 - 3.12 (m, 1H), 3.12 - 3.07 (m, 1H), 3.05 - 2.98 (m, 2H), 2.88 - 2.83 (m, 1H), 2.69 - 2.63 (m, 1H), 2.13 - 2.04 (m, 1H), 1.92 - 1.86 (m, 1H), 1.86 - 1.80 (m, 1H), 1.66 - 1.53 (m, 3H), 1.52 -1.42 (m, 1H), 1.28 - 1.21 (m, 1H), 0.92 (t, J = 7.2 Hz, 3H). ¹⁹F NMR (471 MHz, CDCl₃) δ -71.9. ¹³C NMR (126 MHz, CDCl₃) δ 146.1, 143.4, 135.0, 128.6, 122.8, 120.3, 117.7, 115.2, 113.6, 110.5, 110.0, 109.5, 57.2, 54.1, 50.4, 42.5, 38.9, 33.7, 32.1, 30.1, 28.0, 26.6, 21.0, 12.1. LRMS (APCI+) calcd. For C₂₁H₂₆F₃N₂O₃S⁺ [M+H]⁺ 443.2, found 443.1.

### Example 10. Synthesis of N-methyl-ibogamine-10-carbonitrile 10

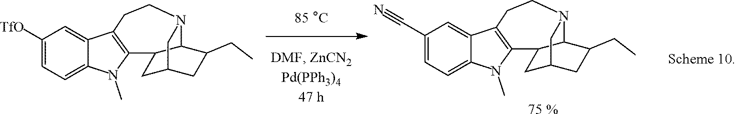

### N-methyl-ibogramine-10-carbonitrile 10

Compound 9 (44 mg, 0.1 mmol), ZnCN₂ (24 mg, 0.2 mmol), Pd(PPh₃)₄ (5.8 mg, 0.005 mmol) were balanced into a reaction vial and dried under vacuum. DMF (anhydrous, 0.5 mL) was added, vial was flushed with argon, closed with a Teflon lined solid screw cap and the RM heated to 85° C. After 24 h, TLC indicated only partial conversion. Additional ZnCN₂ (47 mg, 0.4 mmol) and Pd(PPh₃)₄ (12 mg, 0.01 mmol) were added and heating was continued for additional 23 h, until TLC indicated that no more SM remained. RM was diluted with H₂O (10 mL) and sat. aq. NaHCO₃ solution (2 mL), mixture was extracted with Et₂O (3 × 5 mL) and AcOEt (3 × 5 mL), extraction is complicated by the presence of insoluble solid precipitates. Combined extracts were washed with H₂O, brine, dried over Na₂SO₄, filtered, and concentrated. Crude material was purified by column chromatography (gradient of 0, 5 and 10% of AcOEt in hexanes + 2% Et₃N), followed by PTLC (10% of AcOEt in hexanes + 2% Et₃N). 10 was isolated as a white foamy solid (24 mg, 75%).

¹H NMR (500 MHz, CDCl₃) δ 7.78 (d, J = 1.5 Hz, 1H), 7.36 (dd, J = 8.5, 1.6 Hz, 1H), 7.23 (d, J = 8.5 Hz, 1H), 3.66 (s, 3H), 3.39 - 3.29 (m, 2H), 3.20 - 3.07 (m, 2H), 3.03 - 2.98 (m, 2H), 2.85 (t, J = 1.6 Hz, 1H), 2.73 - 2.66 (m, 1H), 2.12 - 2.05 (m, 1H), 1.91 - 1.80 (m, 2H), 1.64 - 1.51 (m, 3H), 1.51 - 1.44 (m, 1H), 1.27 - 1.21 (m, 1H), 0.91 (t, J = 7.2 Hz, 3H). ¹³C NMR (126 MHz, CDCl₃) δ 145.8, 137.8, 128.3, 123.8, 123.5, 121.4, 110.1, 109.5, 101.5, 57.2, 53.9, 50.3, 42.4, 38.7, 33.6, 32.1, 30.1, 27.9, 26.5, 20.9, 12.0. LRMS (APCI+) calcd. For C₂₁H₂₆N₃⁺ [M+H]⁺ 320.2, found 320.1.

### Example 11. Synthesis of N-methyl-10-ethyl-ibogamine 11

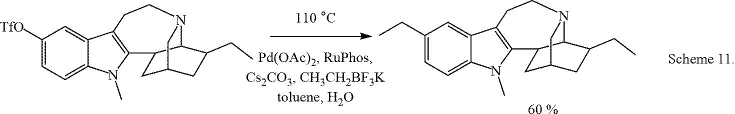

### N-methyl-10-ethyl-ibogamine 11

Compound 9 (44 mg, 0.1 mmol), Pd(Oac)₂ (2.2 mg, 0.01 mmol), RuPhos (8.9 mg, 0.02 mmol), Cs₂CO₃ (98 mg, 0.3 mmol) and CH₃CH₂BF₃K (15 mg, 0.11 mmol) were balanced into a reaction vial. Toluene (not dry, 1 mL) and H₂O (0.1 mL) were added, vial was flushed with argon, closed with a Teflon lined solid screw cap and the RM heated to 110° C. After 62 h, RM was diluted with H₂O (10 mL) and extracted with Et₂O (3 × 5 mL). Combined extracts were dried over Na₂SO₄, filtered, and concentrated. Crude material was purified twice by PTLC (10% of AcOEt in hexanes + 2% Et₃N) and (10% of Et₂O in hexanes + 2% Et₃N). 11 was isolated as a colorless oil (19 mg, 60%).

¹H NMR (500 MHz, CDCl₃) δ 7.31 - 7.28 (m, 1H), 7.15 (d, J = 8.3 Hz, 1H), 7.03 (dd, J = 8.3, 1.7 Hz, 1H), 3.62 (s, 3H), 3.40 - 3.31 (m, 2H), 3.22 - 3.13 (m, 1H), 3.13 - 3.08 (m, 1H), 3.02 (ddt, J = 32.1, 9.3, 2.6 Hz, 2H), 2.86 (t, J = 1.7 Hz, 1H), 2.80 - 2.72 (m, 3H), 2.10 - 2.02 (m, 1H), 1.89 - 1.81 (m, 2H), 1.65 - 1.54 (m, 3H), 1.53 - 1.44 (m, 1H), 1.30 (t, J = 7.6 Hz, 3H), 1.27 - 1.22 (m, 1H), 0.93 (t, J = 7.2 Hz, 3H). ¹³C NMR (126 MHz, CDCl₃) δ 143.5, 134.9, 134.8, 128.5, 121.2, 116.6, 108.6, 108.6, 57.5, 54.4, 50.4, 42.5, 38.8, 33.8, 32.2, 29.8, 29.3, 28.0, 26.7, 21.1, 17.0, 12.1.LRMS (APCI+) calcd. For C₂₂H₃₁N₂⁺ [M+H]⁺ 323.2, found 323.3.

### Example 12. Synthesis of N-ethyl-10-ethoxy-ibogamine 12

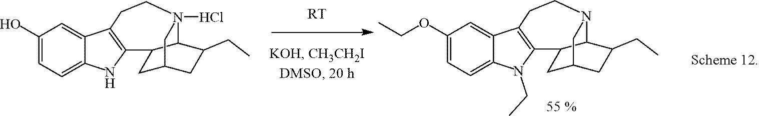

### N-ethyl-10-ethoxy-ibogamine 12

Noribogaine hydrochloride (50 mg, 0.15 mmol) and KOH (53 mg, 0.94 mmol) were mixed in DMSO (anhydrous, 0.3 mL). CH₃CH₂I (36 µL, 0.45 mmol) was added into the suspension and the resulting mixture was stirred at RT for 20 h. Reaction was quenched by adding H₂O (15 mL) and the mixture was extracted with CH₂Cl₂ (3 × 5 mL). Combined extracts were dried over Na₂SO₄, filtered, and concentrated under reduced pressure. Crude material was purified by PTLC (1:60, AcOEt in hexanes + 2% Et₃N, developed twice). 12 was isolated as a yellow waxy solid (29 mg, 55%).

¹H NMR (500 MHz, CDCl₃) δ 7.13 (d, J = 8.7 Hz, 1H), 6.96 (d, J = 2.4 Hz, 1H), 6.82 (dd, J = 8.7, 2.4 Hz, 1 H), 4.28 - 3.90 (m, 4H), 3.45 -3.28 (m, 2H), 3.21 - 3.12 (m, 1H), 3.11 - 3.07 (m, 1H), 3.05 - 2.98 (m, 2H), 2.86 (s, 1H), 2.69 - 2.62 (m, 1H), 2.13 - 2.05 (m, 1H), 1.94 - 1.78 (m, 2H), 1.66 - 1.55 (m, 3H), 1.53 - 1.42 (m, 4 H), 1.31 (t, J = 7.2 Hz, 3H), 1.28 - 1.22 (m, 1H), 0.93 (t, J = 7.2 Hz, 3H). ¹³C NMR (126 MHz, CDCl₃) δ 153.1, 143.5, 130.3, 128.7, 111.2, 109.5, 108.9, 101.9, 64.6, 57.4, 54.5, 50.5, 42.8, 38.6, 37.9, 34.3, 32.1, 28.2, 26.7, 21.0, 16.1, 15.3, 12.1. LRMS (APCI+) calcd. For C₂₃H₃₃N₂O⁺ [M+H]⁺ 353.3, found 353.8.

### Example 13. Synthesis of N-ethyl-noribogaine 13

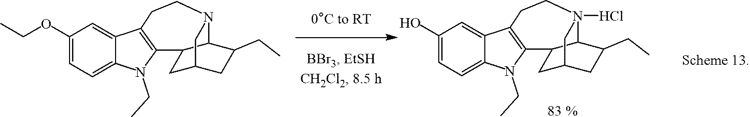

### N-ethyl-noribogaine 13

Compound 12 (68 mg, 0.19 mmol) was dissolved in CH₂Cl₂ (anhydrous, 1.5 mL) and cooled in ice bath. EtSH (0.1 mL, 1.4 mmol) and BBr₃ (1 M in CH₂Cl₂, 0.48 mL) were added, RM was further stirred at RT for 8.5 h and quenched by pouring into sat. aq. NaHCO₃ solution (10 mL). The mixture was extracted with CH₂Cl₂ (3 × 5 mL), combined extracts were dried over Na₂SO₄, filtered, and concentrated under reduced pressure. Crude material was purified by column chromatography (gradient of 1:4, 1:3 to 1:2 AcOEt in hexanes + 2 % Et₃N), excess Et₃N was removed by repeated evaporation from MeOH/H₂O mixture. Still impure free base was transformed to hydrochloride salt by treating its MeOH solution with HCl (36% aq.). The hydrochloride salt was purified by PTLC (5 % MeOH in CH₂Cl₂ + 0.1% HCl (36% aq.), developed twice). 13 was isolated as a yellow amorphous solid (57 mg, 83%).

¹H NMR (500 MHz, MeOD) δ 7.06 (d, J = 8.6 Hz, 1H), 6.78 (d, J = 2.0 Hz, 1 H), 6.64 (dd, J = 8.6, 2.0 Hz, 1H), 4.15 - 3.96 (m, 2H), 3.59 - 3.47 (m, 3H), 3.42 - 3.36 (m, 2H), 3.22 (d, J = 12.3 Hz, 1H), 3.12 -2.98 (m, 2H), 2.31 (t, J = 12.7 Hz, 1H), 2.11 - 2.02 (m, 3H), 1.68 -1.57 (m, 1H), 1.57 - 1.51 (m, 1H), 1.51 - 1.44 (m, 1H), 1.36 - 1.27 (m, 1H), 1.19 (t, J = 7.0 Hz, 3H), 0.97 (t, J = 7.2 Hz, 3H). ¹³C NMR (126 MHz, MeOD) δ 151.6, 140.5, 131.3, 129.1, 112.2, 110.8, 107.7, 103.0, 60.7, 58.2, 52.9, 40.1, 38.6, 33.7, 32.1, 29.6, 27.5, 25.0, 19.4, 16.1, 11.8. LRMS (APCI+) calcd. For C₂₁H₂₉N₂O⁺ [M+H]⁺ 325.2, found 325.6.

### Example 14. Synthesis of N-methyl-10-ethoxy-ibogamine 14

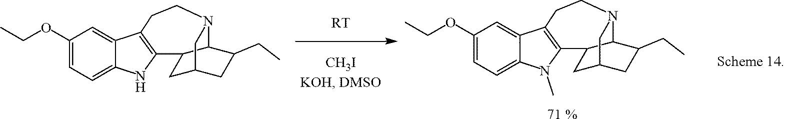

### N-methyl-10-ethoxy-ibogamine 14

10-Ethoxy-ibogamine was prepared as shown in Example 19. 10-Ethoxy-ibogamine (41 mg, 0.13 mmol) and KOH (19 mg, 0.33 mmol) were mixed in DMSO (anhydrous, 0.3 mL). CH₃I (16 µL, 0.26 mmol) was added into the suspension and RM was stirred at RT for 80 min. Reaction was quenched by adding H₂O (6 mL) and the mixture was extracted with Et₂O (3 × 2 mL). Combined extracts were washed with brine (2 mL) dried over Na₂SO₄, filtered, and concentrated under reduced pressure. Crude material was purified by column chromatography (gradient of 0, 5 and 10% Et₂O in hexanes + 2% Et₃N). 14 was isolated as a pale-yellow viscous oil (31 mg, 71%) .

¹H NMR (500 MHz, CDCl₃) δ 7.12 (d, J = 8.7 Hz, 1H), 6.96 (d, J = 2.4 Hz, 1H), 6.84 (dd, J = 8.7, 2.4 Hz, 1H), 4.10 (q, J = 7.0 Hz, 2H), 3.61 (s, 3H), 3.40 - 3.30 (m, 2H), 3.21 - 3.13 (m, 1 H), 3.12 - 3.03 (m, 2H), 3.00 (dt, J = 9.4, 2.8 Hz, 1H), 2.87 (t, J = 1.6 Hz, 1H), 2.73 - 2.66 (m, 1H), 2.11 - 2.03 (m, 1H), 1.90 - 1.81 (m, 2H), 1.66 -1.54 (m, 3H), 1.54 - 1.47 (m, 1H), 1.45 (t, J = 7.0 Hz, 3H), 1.29 -1.22 (m, 1 H), 0.93 (t, J = 7.2 Hz, 3H). ¹³C NMR (126 MHz, CDCl₃) δ 153.1, 144.1, 131.6, 128.5, 111.2, 109.4, 108.6, 101.9, 64.6, 57.4, 54.4, 50.4, 42.5, 38.8, 33.8, 32.2, 29.9, 28.0, 26.7, 21.2, 15.3, 12.1. LRMS (APCI+) calcd. For C₂₂H₃₁N₂O⁺ [M+H]⁺ 339.2, found 338.9.

### Example 15. Synthesis of 9-Bromo-voacangine 15 and 11-Bromo-voacangine 16

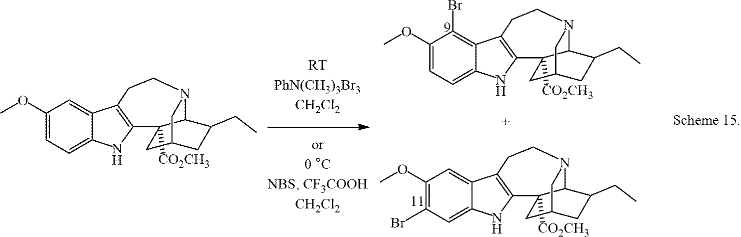

### 9-Bromo-voacangine 15 and 11-bromo-voacangine 16

Voacangine (368 mg, 1.0 mmol) was dissolved in CH₂Cl₂ (anhydrous, 3.7 mL) and phenyltrimethylammonium tribromide - PhN(CH₃)₃Br₃ (414 mg, 1.1 mmol) in CH₂Cl₂ (anhydrous, 5.5 mL) was added dropwise over 30 min. After stirring at RT for 1 h, reaction was quenched by pouring into sat. aq. NaHCO₃ solution (30 mL) and extracted with CH₂Cl₂ (3 × 15 mL) . Combined extracts were dried over Na₂SO₄, filtered, and concentrated under reduced pressure. Crude material was purified by column chromatography (gradient of 10, 15 and 20 % of AcOEt in hexanes) to obtain pure 9-bromo- and a mixed fraction of 9-bromo- and 11-bromo-voacangine. The mixed fraction was separated using PTLC (15% of AcOEt in hexanes, plate developed twice) to obtain 15 as a pale brown solid (260 mg, 58%) and 16 as an off-white amorphous solid (66 mg, 15%).

### 9-Bromo-voacangine 15

¹H NMR (500 MHz, CDCl₃) δ 7.84 (s, 1 H), 7.13 (d, J = 8.7 Hz, 1 H), 6.86 (d, J = 8.7 Hz, 1H), 3.89 (s, 4 H), 3.71 (s, 3 H), 3.63 (s, 1 H), 3.51 -3.43 (m, 1H), 3.42 - 3.35 (m, 1 H), 3.15 - 3.08 (m, 1 H), 2.97 - 2.92 (m, 1H), 2.76 (dt, J = 8.6, 1.8 Hz, 1 H), 2.59 (dt, J = 13.5, 2.1 Hz, 1 H), 1.95 - 1.89 (m, 1 H), 1.89 - 1.84 (m, 1 H), 1.76 - 1.69 (m, 1 H), 1.61 - 1.52 (m, 1 H), 1.49 - 1.39 (m, 1 H), 1.33 (dt, J = 9.5, 6.9 Hz, 1 H), 1.16 - 1.10 (m, 1 H), 0.90 (t, J = 7.4 Hz, 3 H). ¹³C NMR (126 MHz, CDCl₃) δ 175.6, 150.4, 139.2, 132.5, 126.7, 112.2, 110.1, 110.0, 104.0, 58.6, 56.0, 55.4, 53.7, 52.9, 52.8, 38.9, 37.0, 32.1, 27.5, 26.9, 23.0, 11.8. LRMS (APCI+) calcd. For C₂₂H₂₈BrN₂O₃⁺ [M+H]⁺ 477.1, found 477.1.

### 11-Bromo-voacangine 16

¹H NMR (500 MHz, CDCl₃) δ 7.68 (s, 1H), 7.43 (s, 1H), 6.93 (s, 1H), 3.92 (s, 3H), 3.72 (s, 3H), 3.54 (d, J = 1.0 Hz, 1H), 3.44 - 3.33 (m, 1H), 3.24 - 3.10 (m, 2H), 2.99 - 2.89 (m, 2H), 2.79 (dt, J = 8.4, 1.6 Hz, 1H), 2.60 - 2.54 (m, 1H), 1.90 - 1.84 (m, 2H), 1.77 - 1.70 (m, 1H), 1.61 - 1.52 (m, 1H), 1.48 - 1.39 (m, 1H), 1.36 - 1.29 (m, 1H), 1.12 (ddt, J = 12.6, 7.2, 2.2 Hz, 1H), 0.90 (t, J = 7.4 Hz, 3H). ¹³C NMR (126 MHz, CDCl₃) δ 175.6, 150.2, 138.0, 130.9, 128.7, 115.0, 110.4, 107.3, 101.2, 57.6, 57.1, 55.2, 53.1, 52.8, 51.7, 39.2, 36.7, 32.1, 27.5, 26.9, 22.3, 11.8. LRMS (APCI+) calcd. For C₂₂H₂₈BrN₂O₃⁺ [M+H]⁺ 477.1, found 477.1.

Voacangine (368 mg, 1.0 mmol) was dissolved in CH₂Cl₂ (anhydrous, 2.0 mL) and CF₃COOH (2.0 mL), the solution was cooled in ice bath and solid N-Bromosuccinimide (196 mg, 1.1 mmol) was added. Reaction mixture was further stirred for 75 min, quenched by pouring into sat. aq. NaHCO₃ solution (45 mL) and extracted with CH₂Cl₂ (3 × 10 mL). Combined extracts were dried over Na₂SO₄, filtered, and concentrated under reduced pressure. Crude material was purified by column chromatography (1:2 AcOEt in hexanes) to obtain a mixture of 9-Bromo-voacangine and 11-Bromo-voacangine (368 mg, 82%, ratio of 9-Bromo to 11-Bromo 92:8). The regioisomers can be separated as indicated in the above-mentioned reaction.

### Example 16. Synthesis of 9-Bromo-ibogaine 17

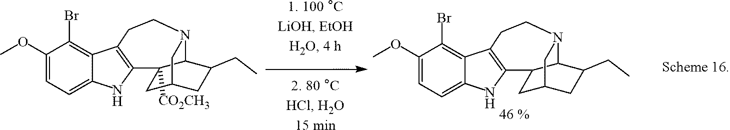

### 9-Bromo-ibogaine 17

To a suspension of 9-bromo-ibogaine (78 mg, 0.17 mmol) in EtOH (0.5 mL) and H₂O (0.25 mL) was added LiOH·H₂O (73 mg, 1.7 mmol) and the RM was heated to 100° C. After 4 h, RM was concentrated, residue was partially dissolved in H₂O (5 mL) and acidified with 2 M aq. HCl (5 mL) to pH ~1. RM was heated to 80° C. for 15 min, cooled to RT and basified with 10% aq. NaOH solution. Mixture was extracted with CH₂Cl₂ (3 × 5 mL), combined extracts were dried over Na₂SO₄, filtered, and concentrated under reduced pressure. Crude material was purified by PTLC (20% AcOEt in hexanes + 2% Et₃N). 17 was isolated as an off-white amorphous solid (36 mg, 46%).

¹H NMR (500 MHz, CDCl₃) δ 7.69 (s, 1H), 7.12 (d, J = 8.6 Hz, 1H), 6.81 (d, J = 8.6 Hz, 1H), 3.89 (s, 3H), 3.70 (dt, J = 16.8, 3.2 Hz, 1H), 3.49 - 3.41 (m, 1H), 3.33 (dt, J = 14.4, 3.5 Hz, 1H), 3.16 - 3.08 (m, 1H), 3.05 (dt, J = 9.7, 2.5 Hz, 1H), 2.99 (dt, J = 9.8, 2.8 Hz, 1H), 2.87 - 2.84 (m, 1H), 2.84 - 2.79 (m, 1H), 2.07 - 1.99 (m, 1H), 1.87 -1.82 (m, 1H), 1.82 - 1.74 (m, 1H), 1.71 - 1.64 (m, 1H), 1.59 - 1.50 (m, 2H), 1.50 - 1.40 (m, 1H), 1.23 - 1.17 (m, 1H), 0.90 (t, J = 7.1 Hz, 3H). ¹³C NMR (126 MHz, CDCl₃) δ 150.2, 145.0, 131.4, 128.0, 111.2, 109.7, 109.0, 103.5, 58.5, 56.6, 54.9, 50.3, 42.1, 42.0, 34.1, 32.1, 28.3, 26.5, 21.4, 12.1. LRMS (APCI+) calcd. For C₂₀H₂₆BrN₂O⁺ [M+H]⁺ 389.1, found 389.7.

### Example 17. Synthesis of 11-Bromo-ibogaine 18

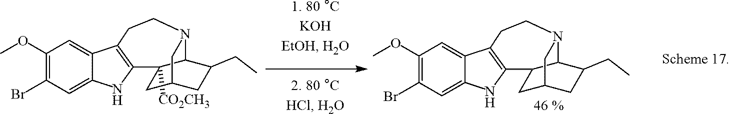

### 11-Bromo-ibogaine 18

To a suspension of 11-bromo-voacangine (30 mg, 0.067 mmol) in EtOH (0.4 mL) and H₂O (0.2 mL) was added KOH (19 mg, 0.34 mmol) and the RM was heated to 80° C. After 48 h, SM was still present, additional KOH (19 mg, 0.34 mmol) was added and heating continued. After 96 h, RM was concentrated, residue was partially dissolved in H₂O (2 mL) and washed with Et₂O (3 × 2 mL). 2 M aq. HCl (2 mL) was added to the H₂O solution and heated to 85° C. for 2 h. RM was cooled to RT and added to 2 M aq. Na₂CO₃ solution (5 mL). Mixture was extracted with CH₂Cl₂ (3 × 5 mL), combined extracts were dried over Na₂SO₄, filtered, and concentrated under reduced pressure. Crude material was purified by PTLC (15% AcOEt in hexanes + 2% Et₃N). 18 was isolated as an off-white amorphous solid (12 mg, 46%).

¹H NMR (500 MHz, CDCl₃) δ 7.52 (s, 1H), 7.41 (s, 1H), 6.94 (s, 1H), 3.92 (s, 3H), 3.41 - 3.28 (m, 2 H), 3.18 - 3.09 (m, 1H), 3.06 (dt, J = 9.3, 2.3 Hz, 1 H), 2.98 (dt, J = 9.3, 2.9 Hz, 1H), 2.91 - 2.83 (m, 2H), 2.64 - 2.50 (m, 1H), 2.08 - 1.99 (m, 1H), 1.87 - 1.76 (m, 2H), 1.68 - 1.60 (m, 1H), 1.59 - 1.50 (m, 2H), 1.50 - 1.41 (m, 1 H), 1.23 - 1.18 (m, 1H), 0.89 (t, J = 7.0 Hz, 3H). ¹³C NMR (126 MHz, CDCl₃) δ 150.0, 143.5, 130.1, 129.7, 114.6, 109.5, 105.9, 100.8, 57.5, 57.1, 54.2, 50.1, 42.1, 41.6, 34.3, 32.2, 28.0, 26.6, 20.8, 12.1. LRMS (APCI+) calcd. For C₂₀H₂₆BrN₂O⁺ [M+H]⁺ 389.1, found 389.1.

### Example 18. Synthesis of 9-Chloro-ibogaine 19

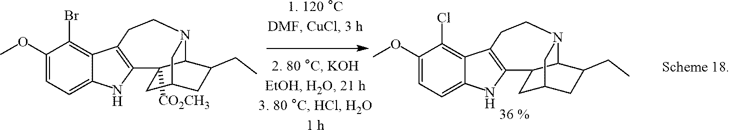

### 9-Chloro-ibogaine 19

9-Bromo-voacangine (67 mg, 0.15 mmol) and CuCl₂ (23 mg, 0.23 mmol) were heated in DMF (anhydrous, 1 mL) to 120° C. for 3 h. After cooling to RT, reaction was diluted with H₂O (15 mL) and extracted with CH₂Cl₂ (3 × 5 mL), combined extracts were washed with H₂O and brine, dried over Na₂SO₄, filtered, and concentrated under reduced pressure. Crude material (~56 mg, not pure) was used for the next reaction without further purification. Solid KOH (39 mg, 0.7 mmol) was added to the suspension of crude material in EtOH (0.8 mL) and H₂O (0.4 mL), and the resulting RM was heated to 80° C. After 21 h, RM was concentrated, residue was partially dissolved in H₂O (2 mL) and acidified with 2 M aq. HCl (3 mL) to pH ~1. RM was heated to 80° C. for 1 h, cooled to RT and slowly basified with 2 M aq. Na₂CO₃ solution (5 mL). Mixture was extracted with CH₂Cl₂ (3 × 5 mL), combined extracts were dried over Na₂SO₄, filtered, and concentrated under reduced pressure. Crude material was purified by column chromatography (gradient of 1:9, 1:5 to 1:4 AcOEt in hexanes + 2% Et₃N) followed by PTLC (25% AcOEt in hexanes + 2% Et₃N). 19 was isolated as an off-white amorphous solid (25 mg, 36% over two steps).

¹H NMR (500 MHz, CDCl₃) δ 7.65 (s, 1H), 7.07 (d, J = 8.6 Hz, 1H), 6.83 (d, J = 8.7 Hz, 1H), 3.90 (s, 3H), 3.59 - 3.52 (m, 1H), 3.52 - 3.44 (m, 1H), 3.35 - 3.29 (m, 1H), 3.17 - 3.09 (m, 1H), 3.02 (ddt, J = 36.5, 9.7, 2.6 Hz, 2H), 2.87 - 2.81 (m, 2H), 2.08 - 2.00 (m, 1H), 1.87 - 1.82 (m, 1H), 1.82 - 1.75 (m, 1H), 1.70 - 1.64 (m, 1H), 1.59 - 1.51 (m, 2H), 1.49 - 1.40 (m, 1H), 1.23 - 1.17 (m, 1H), 0.90 (t, J = 7.1 Hz, 3H). ¹³C NMR (126 MHz, CDCl₃) δ 149.4, 144.7, 131.4, 126.9, 114.3, 110.7, 109.2, 108.9, 58.4, 56.8, 55.0, 50.3, 42.1, 42.0, 34.2, 32.1, 28.3, 26.5, 21.5, 12.1. LRMS (APCI+) calcd. For C₂₀H₂₅ClN₂O⁺ [M+H]⁺ 345.2, found 389.7. LRMS (APCI+) calcd. For C₂₀H₂₅ClN₂O⁺ [M+H]⁺ 345.2, found 345.2.

### Example 19. Synthesis of 10-Ethoxy-ibogamine

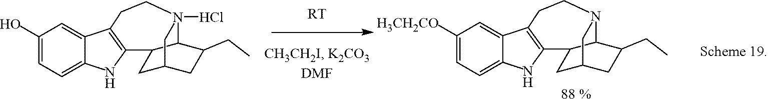

Noribogaine hydrochloride (166 mg, 0.5 mmol) and K₂CO₃ (276 mg, 2.0 mmol) were combined in DMF (anhydrous, 2.0 mL) and CH₃CH₂I (80 µL, 1.0 mmol) was added. Reaction mixture was further stirred 23 h under argon atmosphere and quenched by pouring into H₂O (20 mL). Aqueous mixture was extracted with Et₂O (3 x 10 mL), combined extracts were washed with H₂O (10 mL), brine (10 mL) and dried over Na₂SO₄. Crude material was purified by column chromatography (gradient of 0%, 10%, 20 and 25 % Et₂O in hexanes + 2% Et₃N) . 10-Ethoxy-ibogamine was obtained as a thick colorless oil (143 mg, 88%).

¹H NMR (500 MHz, CDCl₃) δ 7.55 (s, 1H), 7.13 (d, J = 8.6 Hz, 1H), 6.95 (d, J = 2.4 Hz, 1H), 6.78 (dd, J = 8.6, 2.4 Hz, 1H), 4.09 (q, J = 7.0 Hz, 2H), 3.42 - 3.28 (m, 2H), 3.20 - 3.04 (m, 2H), 2.98 (dt, J = 9.4, 3.0 Hz, 1H), 2.94 - 2.81 (m, 2H), 2.66 - 2.55 (m, 1H), 2.09 - 1.98 (m, 1 H), 1.90 - 1.74 (m, 2H), 1.69 - 1.61 (m, 1H), 1.61 - 1.51 (m, 2H), 1.45 (q, J = 8.4, 7.0 Hz, 4H), 1.25 - 1.17 (m, 1H), 0.91 (t, J = 7.0 Hz, 3H). ¹³C NMR (126 MHz, CDCl₃) δ 153.3, 143.0, 130.3, 129.9, 111.4, 110.9, 109.2, 101.8, 64.5, 57.6, 54.3, 50.1, 42.1, 41.7, 34.3, 32.2, 28.0, 26.6, 20.8, 15.3, 12.1. LRMS (APCI+) calcd. For C₂₁H₂₉N₂O⁺ [M+H]⁺ 325.2, found 325.2

### Biological Characterization of Iboga Analogs

### Example 20. Cell Culture Preparation and Maintenance

Stably transfected hSERT-HEK and rVMAT2-HEK cellular cultures were maintained in Dulbecco’s Minimal Essential Medium (DMEM) with GlutaMAX (Gibco) with the following additions: 10% (v/v) Fetal Bovine Serum (FBS, Atlanta Biologicals), 100 U/mL Penicillin, and 10 µg/mL Streptomycin (Gibco). With regards to the former cell lineage, an additional ingredient, 500 µg/mLGeneticin (G418) (Sigma) was included to preserve the respective transgene.

### Example 21. hSERT and rVMAT2 Screening Assays

For both hSERT and rVMAT2 screening experiments, respective singly transfected cells were seeded at a density of 0.09 × 10⁶ cells/well in poly-D-Lysine (Sigma) coated white solid-bottom 96-well plates. Growth was permitted for approximately 44 hours in said aqueous media and at an incubation environment of 37° C. and 5% Carbon Dioxide. At the beginning of the experiment, the cellular growth solution was aspirated, and individual cells were rinsed with 150 µL of 1× Dulbecco’s Phosphate Buffered Saline (PBS; HyClone). 63 µL of Experimental Media (consisting of the following contents: DMEM without phenol red but with 4.5 g/L of D-Glucose (Gibco), 1 % (v/v) FBS (Atlanta Biologicals), 100 U/mL Penicillin, and 10 µg/mLStreptomycin (Gibco)) with 2 × tiered concentrations of inhibitor (or DMSO, the vehicle of these experiments) were added to the respective wells. Control inhibitors used in these studies include Imipramine for hSERT experiments, and Reserpine for rVMAT2 experiments (Eiden, L. E. and Weihe, E. 2011; Sette, M. et al. 1983). At the conclusion of the one hour long pre-incubation period, 63 µL of Experimental Media containing 2 × various concentrations of tested inhibitor (or vehicle) along with a specified amount of fluorescent substrate, APP⁺ (Karpowicz, R. J. et al 2013) (final concentration: 1.1 µM for hSERT experiments) or FFN206 (Hu, G. et al. 2013) (final concentration: 0.75 µM for rVMAT2 experiments) were added to the present solution contained within the wells. After a required incubation period of 30 minutes for proper fluorescent probe uptake, the contents of each well were aspirated and consequently, rinsed twice with 120 µL of PBS. A final solution of 120 µL of PBS is finally added to all corresponding wells for cell maintenance before undergoing fluorescence uptake reading by a BioTek H1MF plate reader. The excitation and emission wavelengths of APP⁺ were set at 389 and 442 nm, respectively. Alternatively, the excitation and emission wavelengths of FFN206 were designed at 370 and 464 nm, respectively.

### Data Analysis

Numerical analysis of the collected experimental data preceded as accordingly. Respective inhibitor values were first subtracted from vehicular values to quantify the respective fluorescence uptake. This metric was then analyzed using the dose-response-inhibitor nonlinear curve fitting model ([inhibitor] vs response (three parameters)) as supplied by GraphPad Prism 6 software. For each inhibitor, the model supplied a respective IC₅₀ ± SEM value (Table 1). From this intermediate metric, calculation of the inhibition constant, Kᵢ ± SEM, was made possible using the Cheng-Prusoff Equation (Yung-Chi, C. and Prusoff, W. H. 1973) and the following established constants: Kₘ (for APP⁺) = 1.6 µM (hSERT) and Kₘ (for FFN206) = 1.2 µM (rVMAT2). It must be noted that the lower the Kᵢ value that is found, the greater the potency that the candidate inhibitor possesses at said transporter.

TABLE 1

Selected Notable Iboga Analogs IC₅₀ Values for hSERT and rVMAT2 Transporters

| | SERT [µM ± SEM] | VMAT2 [µM ± SEM] |
|---|---|---|
| Ibogaine | ~ 0.5 - 10 | ~ 4 |
| Noribogaine | 0.28 ± 0.04 | 0.57 ± 0.08 |
| 10-Ethoxy-ibogamine | 9.0 ± 1.1 | 0.08 ± 0.02 |
| 2 | 2.5 ± 0.29 | 3.0 ± 0.57 |
| 3 | 0.026 ± 0.0038 | 3.3 ± 0.43 |
| 4 | 3.0 ± 0.50 | |
| 5 | 0.33 ± 0.04 | 3.0 ± 0.55 |
| 6 | 0.08 ± 0.009 | 1.5 ± 0.16 |
| 7 | 1.5 ± 0.13 | 0.74 ± 0.11 |
| 8 | 0.059 ± 0.011 | 0.17 ± 0.03 |
| 10 | 0.0051 ± 0.00083 | 0.44 ± 0.05 |
| 11 | 0.91 ± 0.12 | 0.75 ± 0.07 |
| 13 | 1.9 ± 0.20 | 0.07 ± 0.01 |
| 14 | 2.8 ± 0.38 | |
| 17 | 0.67 ± 0.16 | 4.9 ± 0.87 |
| 18 | 0.20 ± 0.03 | 1.0 ± 0.15 |
| 19 | 1.2 ± 0.12 | 18 ± 5.9 |

All data is expressed in µM concentrations, and express the average values as calculated from experiments n ≥ 4, with corresponding ± SEM. Note that literature values for respective serotonergic binding inhibition capability of Ibogaine are listed above (Mash, D. C. et al. 1995).

### Examples 22-31. Additional Ibogaine and Noribogaine Analogs

Additional analogs of ibogaine and noribogaine are prepared using methods described throughout this application and methods available to individuals skilled in the art. Compounds 20-43, 44a and 44b are synthesized according to Schemes 20-29.

### Example 22. Synthesis of 9-chloro-noribogaine 20 and 9-iodo-ibogaine 21

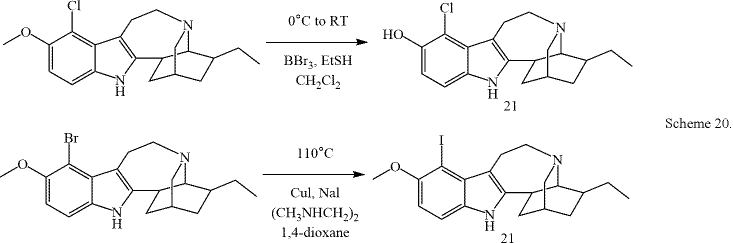

### Example 23. Synthesis of 11-halo-ibogaine and Noribogaine Derivatives 22-24

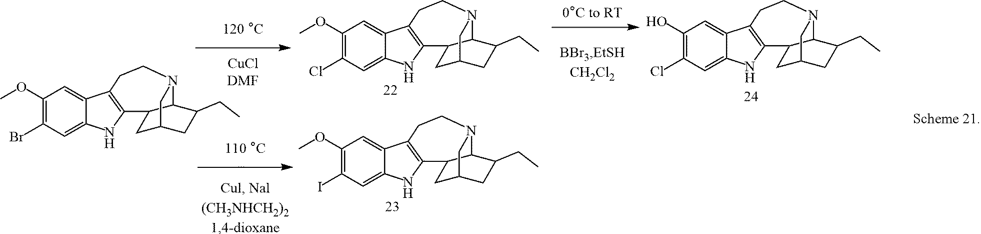

### Example 24. Synthesis of 12-methyl-ibogaine 25 and 12-methyl-noribogaine 26

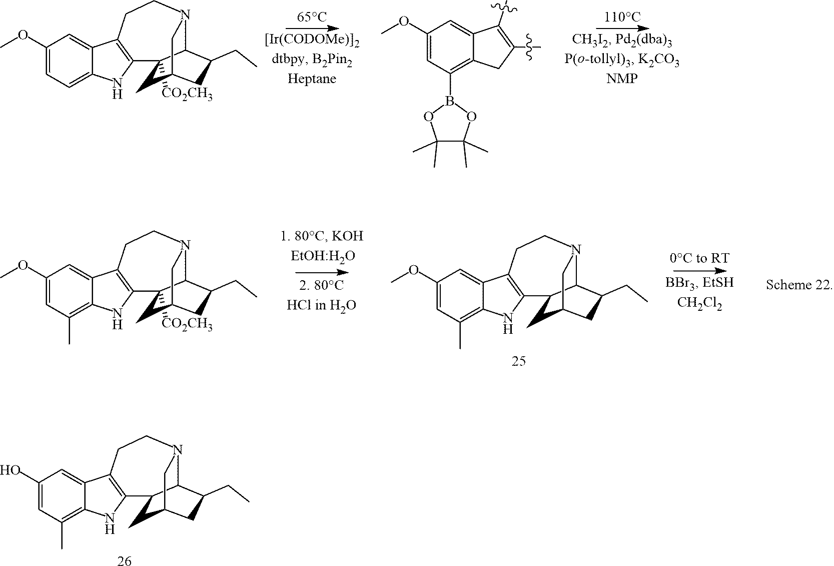

### Example 25. Synthesis of Derivatives 27 and 28

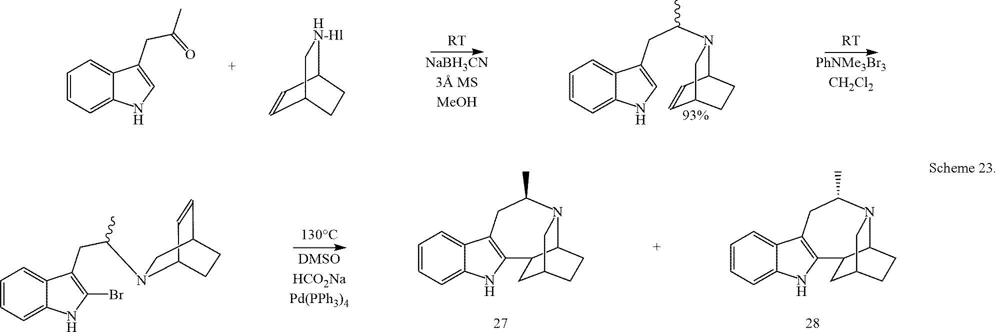

### Example 26. Synthesis of Derivative 29

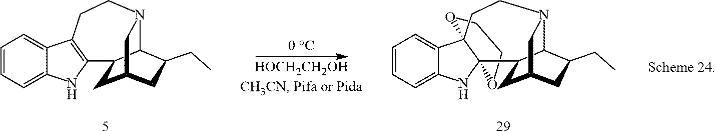

### Example 27. Synthesis of 9-bromo-noribogaine 30 and 11-bromo-noribogaine 31

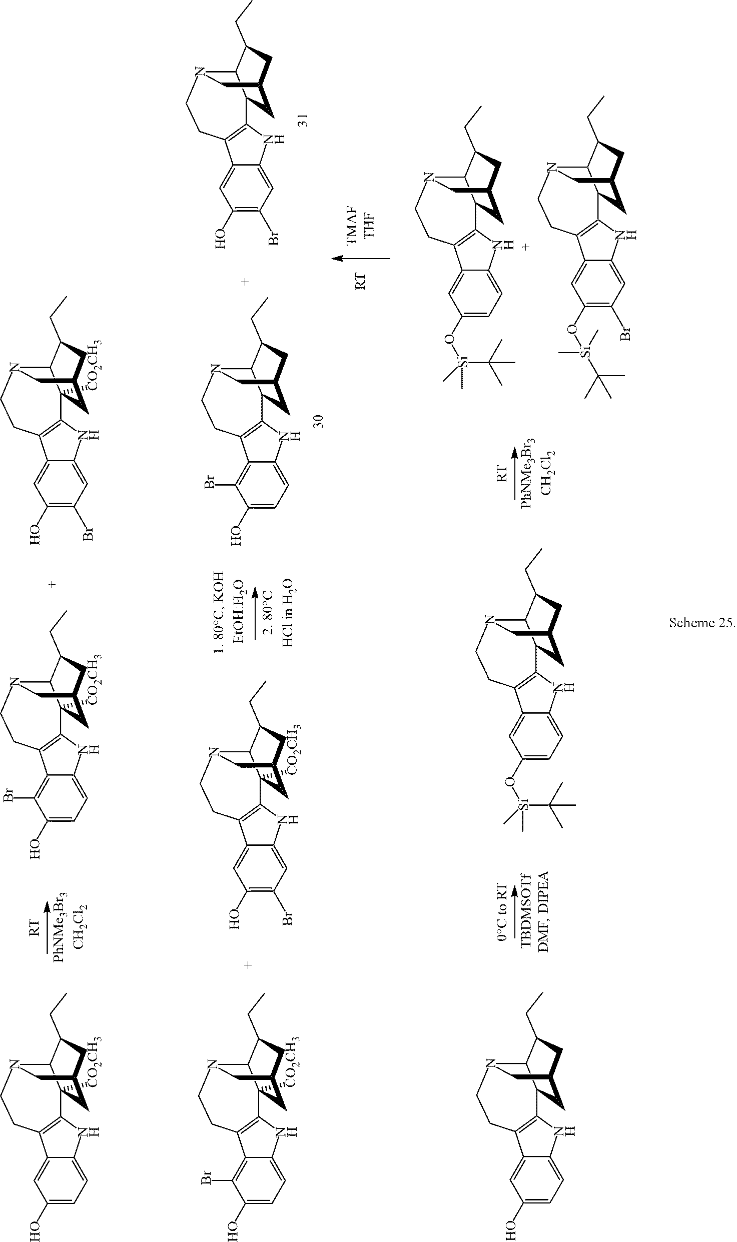

### Example 28. Synthesis of 9-amino, -carboxamido, -cyano and -hydroxy ibogaine Derivatives 32-35

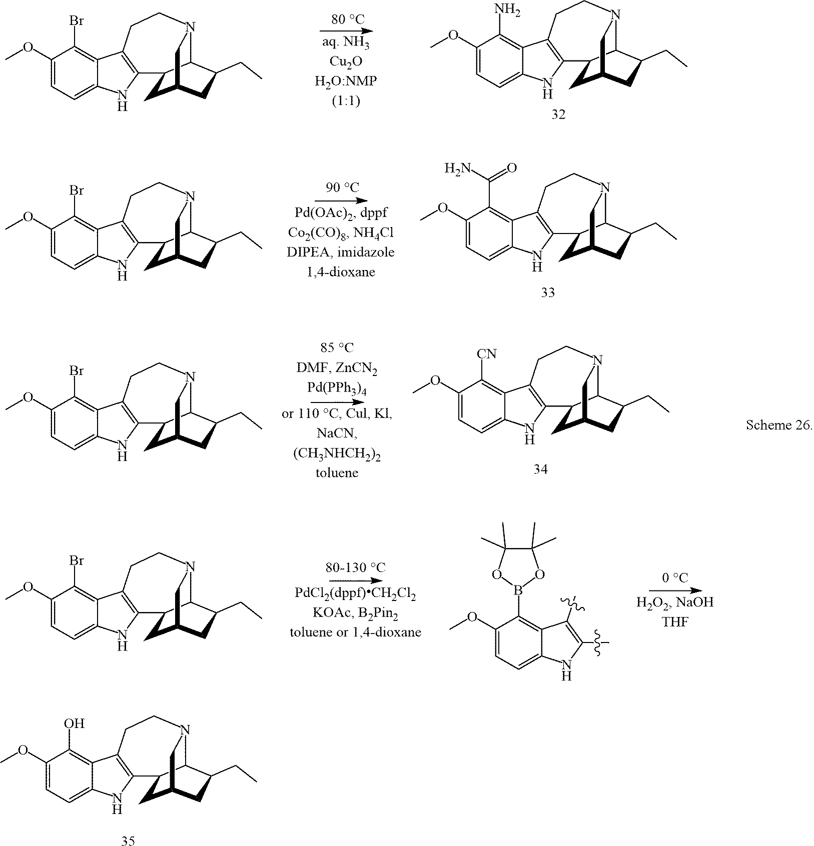

### Example 29. Synthesis of 9-fluoro-ibogaine 36 and N-methyl-9-fluoro-ibogaine 37

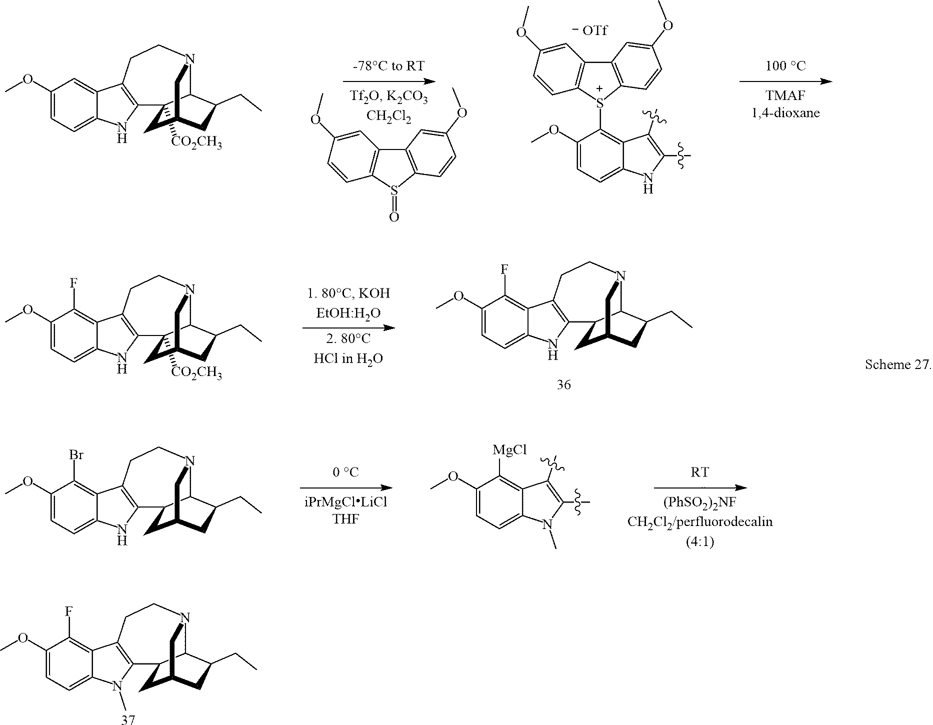

### Example 30. Synthesis of 10-bromo, -iodo, -trifluoromethyl, -chloro, and -fluoro ibogamine Derivatives 38-42

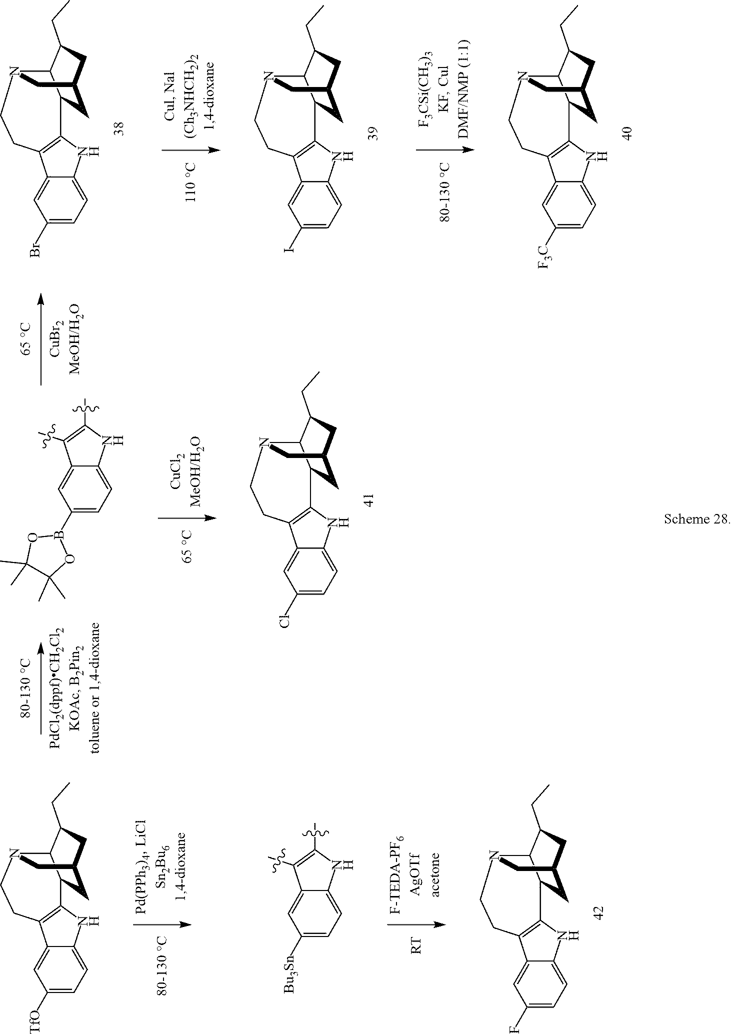

### Example 31. Synthesis of N-methyl-ibogaine Derivatives 43a and 43b

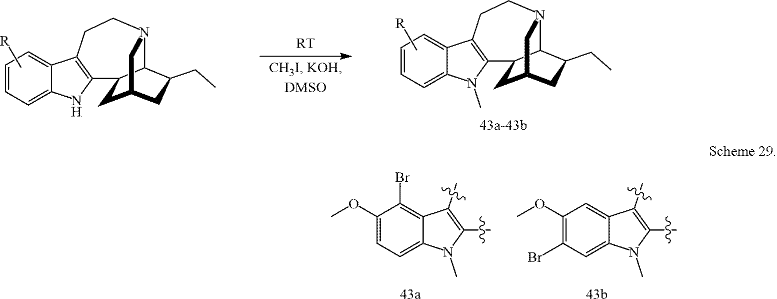

Metabolism Studies. Metabolic stability of test compounds was evaluated in liver microsomes from rat (RLM) and human (HLM). This was accomplished by incubating test compounds with microsomes and monitoring disappearance of parent compound as well as metabolite formation over time using LC-MS/MS in Multiple Reaction Monitoring mode (MRM). Verapamil in HLM and RLM was used as positive control. Pooled human liver microscomes or pooled male rats liver microsomes (20 mg/mL) were diluted in Kphos buffer to prepare a concentration of 0.714 mg/mL.

### Preparation of Test Compounds

Stock solutions of test compounds were prepared in DMSO at a concentration of 10 mM.

Assay Conditions

| | |
|---|---|
| Total Incubation volume | 100 µL |
| Compound concentration | 10 µM |
| Protein Concentration controls) | 0.5 mg/mL (0.25 mg/mL for positive |
| NADPH | 1 mM |
| Final DMSO content | 0.1% |
| Number of replicates | 2 |
| Time points | 0, 5, 15, 30 and 60 min |

### Assay

An 1120 µL aliquot of Kphos buffer (50 mM, pH 7.4) containing liver microsomes (0.714 mg/mL) were added to individual 2 mL tubes (final concentration 0.5 mg/mL). Test compounds (10 mM) and positive control were directly spiked into respective tubes to prepare a concentration of 14.28 µM (final concentration 10 µM). From the above mix, 70 µL was added to individual wells of 96 well reaction plates and preincubated at 37° C. for 5 min. All the reactions were initiated by adding 30 µL of 3.33 mM NADPH (final concentration 1 mM). Reactions without NADPH and buffer controls (minus NADPH) at 0 min and 60 min were also incubated to rule out non-NADPH metabolism or chemical instability in the incubation buffer. All reactions were terminated using 100 µL of ice-cold acetonitrile containing internal standard (glipizide) at 0, 5, 15, 30 and 60 min. The plates were centrifuged at 4000 RPM for 15 min and 100 µL aliquots were submitted for analysis by LC-MS/MS.

### Preparation of Calibration Curve

Stock solution of noribogaine was prepared in DMSO at a concentration of 20 mM and serially diluted in DMSO to prepare 10, 3, 1, 0.3, 0.1 and 0.03 mM solutions respectively. These were diluted 1000 folds in microsomes to attain final concentration of 20, 10, 3, 1, 0.3, 0.1 and 0.03 µM respectively.

### Bio-Analysis

Samples were monitored for parent compounds disappearance and formation of metabolite in MRM mode using LC-MS/MS instrument (API-4000 with Waters UPLC).

LC-MS/MS conditions

| Mobile Phase | A: 0.1 % Formic acid in Acetonitrile B: 10 mM Ammonium formate in water |
|---|---|
| Column | Accucore C8, 50 × 2.1 mm, 2.6 µm |
| Injection Volume (µL) | 1 |
| Column Oven Temperature (°C) | 45 |

LC Gradient Used

| Time (min) | Flow (mL/min) | PUMP A (% Conc.) | PUMP B (% conc .) |
|---|---|---|---|
| Initial | 0.6 | 0 | 100 |
| 0.3 | 0.6 | 0 | 100 |
| 0.5 | 0.6 | 95 | 5 |
| 1.4 | 0.6 | 95 | 5 25 |
| 1.8 | 0.6 | 0 | 100 |
| 2.2 | 0.6 | 0 | 100 |

Retention Times and MRM Transitions in Positive Polarity Mode

| Analyte ID / IS ID | Retention time (min) | Q1 | Q3 | DP | CE | CXP | Dwell time (msec) |
|---|---|---|---|---|---|---|---|
| Noribogaine | 1.05 | 297.7 | 160 | 114 | 45 | 11 | 10 |
| Ibogaine | 1.08 | 311.9 | 122.2 | 72 | 46 | 22 | |
| EtO-Ibogamine | 1.10 | 325.1 | 188.2 | 100 | 42 | 14 | |
| Glipizide | 1.05 | 446.3 | 347.0 | 40 | 22 | 12 | |
| Verapamil | 0.94 | 455.5 | 165.5 | 105 | 42 | 11 | |

Source Parameters

| Source Parameter | Parameter value/ description |
|---|---|
| Polarity | Positive |
| CAD | 8 |
| CUR | 30 |
| GS1 | 40 |
| GS2 | 60 |
| Ion Spray Voltage | 5500 |
| Temperature | 550 |
| Interface Heater | ON |
| EP | 10 |

### Data Analysis

The percent remaining of test compounds and positive control in each sample was determined by considering peak area ratio in the 0 minute sample as 100%. The Half-life of compounds in microsomes is calculated by formula:

Half-life (t_{½}) (min) = 0.693/k, where k is gradient of line determined from plot of peak area ratio (compound peak area / internal standard peak area) against time.

In vitro intrinsic clearance (CL´ᵢₙₜ) (units in mL/min/kg) was calculated using the formula:

$$\begin{matrix}C {{L}^{′}}_{\text{int}} = \frac{0.693}{\text{in} \text{vitro} \text{T1} / 2} ⋅ \frac{\text{mL incubation}}{\text{mg microsomes}} ⋅ \frac{\text{45 mg microsomes}}{\text{gm liver}} ⋅ \\ \frac{\text{liver weight in gm*}}{\text{Kg b} \text{.w}}\end{matrix}$$

For liver microsomes, scaling factor used was 45 mg microsomal protein per gm liver.

* Indicates liver weight (gm) which varies species wise. For human, monkey, dog, rat and mouse the liver weight are 20 gm, 32 gm, 32 gm, 40 gm and 90 gm respectively.

### Example 32. Metabolism of 10-Ethoxy-ibogamine

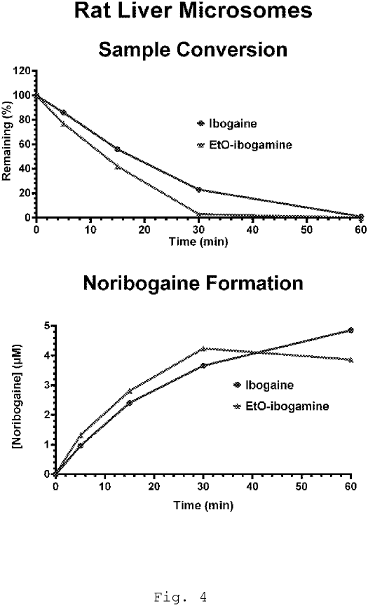

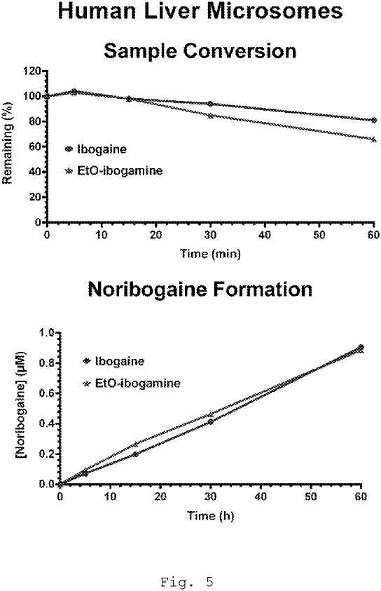

The metabolic degradation of 10-ethoxy-ibogamine in human liver microsomes (HLM) to noribogaine occurs at a rate that is similar to ibogaine (Scheme 30, Tables 2-3, and FIGS. 4-5).

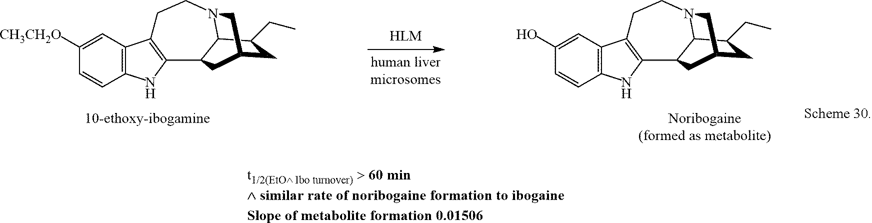

TABLE 2

Percentage turnover of test compounds in rat (RLM) and human liver microsomes (HLM)

| Time (min) | Verapamil | | Ibogaine | | Eto-Ibogamine | |
|---|---|---|---|---|---|---|
| | RLM | HLM | RLM | HLM | RLM | HLM |
| 0 | 100 | 100 | 100 | 100 | 100 | 100 |
| 5 | 72 | 67 | 86 | 104 | 77 | 103 |
| 15 | 51 | 49 | 56 | 98 | 42 | 98 |
| 30 | 35 | 38 | 23 | 94 | 3 | 85 |
| 60 | 20 | 21 | 1 | 81 | 0 | 66 |
| % Remaining at 60 min (+ NADPH) | 20 | 21 | 1 1 | 81 | 0 | 66 |
| % Remaining at 60 min (- NADPH) | 98 | 104 | 104 | 98 | 88 | 106 |
| %Remaining at 60 min (Buffer) | 120 | | 103 | | 88 | |
| t_{½} (min) | 27 | 29 | 8 | >60 | 6 | >60 |
| CL_{h,int}(mL/min/kg) | 185 | 85 | 306 | 7 | 445 | 13 |

TABLE 3

Concentrations (µM) of noribogaine detected in samples of ibogaine and Eto-ibogamine in the course of microsomal transformation

| Time (min) | Ibogaine Ibogaine | | Eto-Ibogamine | |
|---|---|---|---|---|
| | RLM | HLM | RLM | HLM |
| 0 | 0.000 | 0.000 | 0.02 | 0.000 |
| 5 | 0.960 | 0.071 | 1.320 | 0.095 |
| 15 | 2.405 | 0.198 | 2.810 | 0.266 |
| 30 30 | 3.660 | 0.412 | 4.240 | 0.464 |
| 60 | 4.855 | 0.906 | 3.865 | 0.886 |

### Discussion

Minor structural modifications of ibogaine or noribogaine have large and unexpected effects on the pharmacological activity, particularly at monoamine transporters. The modulation of the serotonin transporter (SERT) has been invoked as one of the key molecular mechanisms contributing to the ibogaine’s in vivo effects (Baumann, M.H. et al. 2001; Staley, J. K. et al. 1996; Jacobs, M. T. et al. 2007). Furthermore, vesicular monoamine transporter 2 (VMAT2) has been shown as a target for the treatment of SUDs (Nickell, J. R. et al. 2010). We here show that modulation of the vesicular monoamine transporter 2 (VMAT2) contributes to ibogaine’s effects, and further show that for some compounds dual inhibition of SERT and VMAT2 is mechanistically important.

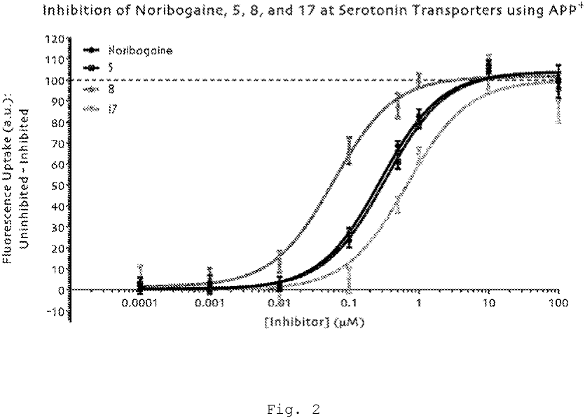

The results of the SERT inhibition assay using the fluorescent substrate, APP⁺, for ibogaine, noribogaine and Compounds 2-19 are shown in Table 1. Potent inhibition of SERT is seen for noribogaine and respective analogs (Table 1 and FIG. 2). Noribogaine possesses the following values: IC₅₀ = 0.28 ± 0.04 µM and Kᵢ = 0.17 ± 0.02 µM, which is comparable to corresponding data as stated previously (Wasko, M. J. et al. 2018). Compound 5 is similar in potency to noribogaine as evidenced by its values: IC₅₀ = 0.33 ± 0.04 µM and Kᵢ = 0.20 ± 0.02 µM. Compound 8 is approximately 4.7 times more potent than noribogaine (IC₅₀ = 0.059 ± 0.011 µM and Kᵢ = 0.035 ± 0.0067 µM) while Compound 17 is approximately 2.4 times less potent comparatively (IC₅₀ = 0.67 ± 0.16 µM and Kᵢ = 0.40 ± 0.09 µM).

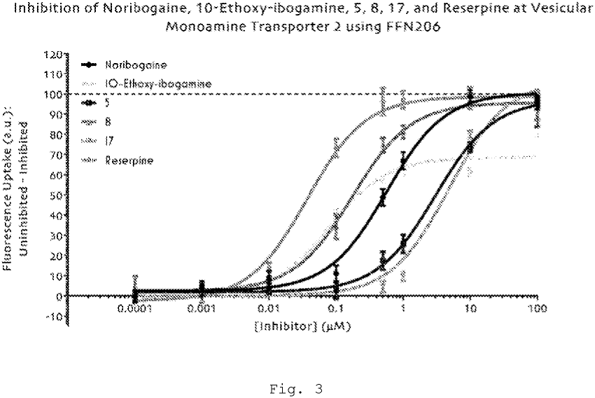

The results of the VMAT2 inhibition assay using the fluorescent substrate, FFN206, for ibogaine, noribogaine and Compounds 2-19 are shown in Table 1. Potent inhibition of VMAT2 is seen for noribogaine and respective analogs (Table 1 and FIG. 3). Noribogaine possesses the following values: IC₅₀ = 0.57 ± 0.08 µM and Kᵢ = 0.35 ± 0.04 µM. 10-Ethoxy-ibogaine has an IC₅₀ value of 0.08 ± 0.02 µM and a Kᵢ of 0.05 ± 0.01 µM, being nearly an order of magnitude more potent at this target. Compound 8 (IC₅₀ = 0.17 ± 0.03 µM and Kᵢ = 0.10 ± 0.02 µM) is approximately 3.4 times more potent than noribogaine. Compounds 5 (IC₅₀ = 3.0 ± 0.55 µM and Kᵢ = 1.8 ± 0.34 µM) and 17 (IC₅₀ = 4.9 ± 0.87 µM and Kᵢ = 3.0 ± 0.53 µM) are approximately 5.3 and 8.6 times weaker than noribogaine respectively. Reserpine, a recognized VMAT2 inhibitor, was included in this analysis as a control and possess the following values: IC₅₀ = 0.017 ± 0.0055 µM and Kᵢ = 0.011 ± 0.0031 µM (Eiden, L. E. and Weihe, E. 2011).

The data herein shows that some compounds selectively inhibit VMAT2, while other compounds selectively inhibit SERT, and still other compounds inhibit both VMAT2 and SERT. The data here also shows the unexpected effect of 10-ethoxy-ibogamine on monoamine transporters. 10-Ethoxyibogamine is a potent VMAT2 inhibitor but is only a weak inhibitor of SERT. In contrast, noribogaine, which can be formed by the metabolism of 10-ethoxy-ibogamine (FIGS. 4-5), is a potent SERT inhibitor. Therefore, we can achieve dual modulation of VMAT2 and SERT through a combined effect of the administered drug and its metabolite.

The serotonin transporter (SERT) and vesicular monoamine transporter 2 (VMAT2) are important targets for the treatment of psychiatric and neurological disorders. The compounds described herein are shown to possess properties useful for the treatment of indications described above.

### REFERENCES

Alper, K.R. et al. Am J Addict 8, 234-242 (1999).

Alper, K.R. Ibogaine: A Review. Alkaloids Chem Biol 56, 1-38 (2001).

Baumann, M.H. et al. The Journal of Pharmacology and Experimental Therapeutics 297, 531-539 (2001).

Belgers, M. et al. Transl Psychiatry 6, e826 (2016).

Bhargava, H.N. & Cao, Y-J. Brain Research 771, 343-346 (1997).

Brown, T.K. & Alper, K. Am J Drug Alcohol Abuse 44, 24-36 (2018).

Brown, T.K. et al. J Psychoactive Drugs 51, 155-165, (2019).

Dean, M. & Sung, V.W. Drug Design, Development and Therapy 12, 313-319, (2018).

Eiden, L.E. & Weihe, E. Ann N Y Acad Sci 1216(1), 86-98 (2011).

He, D-Y. et al. The Journal of Neuroscience 25(3), 619-628 (2005).

Gash, D.M. et al. Nature 380, 252-255 (1996)

Glick, S.D. et al. Alkaloids Chem Biol 56, 39-53 (2001).

Glue, P. et al. Clinical Pharmacology in Drug Development 5(6), 460-468 (2016).

Glue, P. et al. The Journal of Clinical Pharmacology 55(6), 680-687 (2015).

Gonzalez, J. et al. Front. Pharmacol. 9, 374 (2018).

Hu, G. et al. ACS Chemical Biology 8(9), 1947-1954 (2013).

Jacobs, M.T. et al. J Biol Chem 282, 29441-29447 (2007).

Jankovic, J. et al. Tremor Other Hyperkinet. Mov. (N Y). 6(422), (2016).

Karpowicz, R.J. et al. ACS Chem. Neurosci. 4(5), 858-869 (2013).

Kroupa, P.K. & Wells, H. MAPS, XV(2), 21-24 (2005).

Marton, S. et al. Front. Pharmacol. 10, 193 (2019)

Mash, D.C. et al. Ann N Y Acad Sci 844, 274-292 (1998).

Mash, D.C. et al. Ann N Y Acad Sci 914, 394-401 (2000).

Mash, D.C. et al. J Psychopharmacol 30, 688-697 (2016).

Mash, D.C. et al. Front Pharmacol 9, 529 (2018).

Mash, D.C. et al. Life Sciences 57(3), (1995).

Naranjo, C. The Healing Journey. (Random House, 1973).

Nickell, J.R. et al. J. Pharmacol. Exp. Ther. 332, 612-621 (2010).

Noller, G.E. et al. Am J Drug Alcohol Abuse 44, 37-46 (2018).

Rodriǵuez, P. et al. ACS Chem. Neurosci. 11(11), 1661-1672 (2020).

Rubi, L. et al. Cardiovascular Toxicology 17, 215-218(2017).

Schenberg, E.E. et al. J Psychopharmacol 28, 993-1000 (2014).

Schneider, J.A. & Sigg, E.B. Ann N Y Acad Sci 66, 765-776 (1957).

Sette, M. et al. Journal of Neurochemistry 40(3), 622-628 (1983).

Sharma, S.S. & Bhargava, H.N. Pharmacology 57, 229-232 (1998).

Shear, M.K. et al. Depress Anxiety 28(2), 103-117 (2011).

Solmi, M. Drug Design, Development and Therapy 12, 1215-1238 (2018). Sreeram, V. et al. Cureus 11(8), e5471 (2019).

Staley, J.K. et al. Psychopharmacology (Berl) 127, 10-18 (1996).

Thoricatha, W. Psychedelic Times Jan. 28, 2020.

Wasko, M.J. et al. ACS Chem. Neurosci. 9(10), 2475-2483 (2018).

Yung-Chi, C. & Prusoff, W.H. Biochemical Pharmacology 22(23), 3099-3108 (1973).

What is claimed is:

1. A compound having the structure: 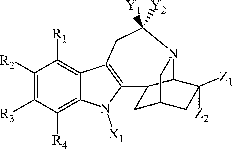
wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
wherein each Y₃ is, independently, —OH, -O(alkyl), —NH₂, -NH(alkyl) or halogen, and each Y₄ is, independently, —OH, O(alkyl),NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
wherein each Z₃ is, independently, —OH, -O(alkyl), —NH₂, -NH(alkyl) or halogen, and each Z₄ is, independently, —OH, O(alkyl),NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, —OCF₃, -(alkyl), -(alkenyl), -(alkynyl), (aryl),(heteroaryl), —OH, —OAc, -O-(alkyl), -O-(alkenyl), -O- (alkynyl), -O- (aryl), -O- (heteroaryl), —SH, -S-(alkyl), -S-(alkenyl), -S-(alkynyl), -S-(aryl), -S-(heteroaryl), —NH₂, -NH-(alkyl), -NH-(alkenyl), -NH- (alkynyl), -NH- (aryl), -NH-(heteroaryl), -C (O) R₅, -S (O) R₅, -SO₂R₅, -NHSO₂R₅, -OC (O) R₅, -SC(O) R₅, -NHC (O) R₆ or -NHC (S) R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂,
wherein the compound is other than any of ibogaine, ibogamine, N-methyl-ibogaine, N-methyl-noribogaine, N-ethyl-noribogaine, N-methyl-ibogamine or 10-ethoxy-ibogamine,
or a pharmaceutically acceptable salt of the compound.

2. The compound of claim 1, wherein when X₁ is H, then R₁, R₂, R₃ and R₄ are each, independently, —F, —NO₂, —CN, —CF₃, —CF₂H, —OCF₃, -(alkyl), -(alkenyl), -(alkynyl), -(aryl), -(heteroaryl), -O-(alkenyl), -O-(alkynyl), -O-(aryl), -O-(heteroaryl), -S-(alkyl), -S-(alkenyl), S-(alkynyl), -S-(aryl), S- (heteroaryl), C(O)R₅, -S(O)R₅, -SO₂R₅, -NHSO₂R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N(alkyl)₂; or
wherein when X₁ is H, then R₂ is —F, —NO₂, —CN, —CF₃, —CF₂H, -OCF₃, -(alkyl), -(alkenyl), -(alkynyl), -(aryl), -(heteroaryl), O-(alkenyl),O-(alkynyl), -O-(aryl), -O-(heteroaryl), -S-(alkyl), -S-(alkenyl), S-(alkynyl), -S-(aryl), -S-(heteroaryl), C(O)R₅, -S(O)R₅, -SO₂R₅, -NHSO₂R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N(alkyl)₂ and
wherein each R₆ is, independently, -(alkyl), -(aryl), -O-(alkyl), -S-(alkyl), -S-(aryl), —NH₂, -NH(alkyl) or -N(alkyl)₂; or
wherein when X₁, Y₁, Y₂, Z₂, R₁, R₃ and R₄ are each H and Z₁ is ethyl, then R₂ is —F, —NO₂, —CN, —CF₃, —CF₂H, —OCF₃, -(alkyl), -(alkenyl), -(alkynyl), -(aryl), -(heteroaryl), -0- (alkenyl), -O-(alkynyl), -O-(aryl), -O-(heteroaryl), -S-(alkyl), -S-(alkenyl), S- (alkynyl), -S- (aryl), -S- (heteroaryl), -C(O)R₅, -S(O)R₅, -SO₂R₅, -NHSO₂R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N(alkyl)₂; or
wherein when X₁ is alkyl, then R₂ is —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, —OAc, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N(alkyl)₂; or
wherein one of R₁, R₃ and R₄ is —F, —Cl, —Br, —I, —NO₂, —CN, —CF₃, —CF₂H, —OCF₃, -(alkyl), -(alkenyl), -(alkynyl), -(aryl), -(heteroaryl), —OH, —OAc, -O-(alkyl), -O-(alkenyl), -O-(alkynyl), O-(aryl), O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH- (heteroaryl), -C(O)R₅, S(O)R₅, -SO₂R₅, -NHSO₂R₅, -OC(O)R₅, -SC(O)R₅, -NHC (O) R₆ or -NHC (S) R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), -O-(alkyl), -S-(alkyl), -S-(aryl), —NH₂, -NH(alkyl) or -N (alkyl) ₂, and
the others are —H; and
R₂ is —H, —F, —Cl, —Br, —I, —NO₂, —CN, —CF₃, —CF₂H, —OCF₃, -(alkyl), -(alkenyl), -(alkynyl), -(aryl), -(heteroaryl), —OH, —OAc, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O)R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N (alkyl) ₂, and
wherein each R₆ is, independently, -(alkyl), -(aryl), —O—(alkyl), -S-(alkyl), -S-(aryl), —NH₂, -NH(alkyl) or -N(alkyl)₂.

3-6. (canceled)

7. The compound of claim 2 having the structure: 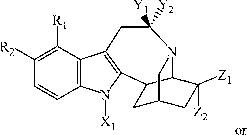 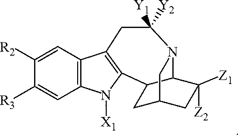
.

8. The compound of of claim 7, wherein
Y₁ is H, -alkyl, -alkenyl, -alkynyl, alkylaryl, -cycloalkyl, -aryl, heteroaryl, -alkyl-Y₃ or -alkyl-C(O)Y₄, and Y₂ is H, -alkyl, -alkenyl, -alkynyl, alkylaryl, -cycloalkyl, -aryl, heteroaryl, -alkyl-Y₃ or -alkyl-C(O)Y₄,
wherein each Y₃ is, independently, -O(alkyl), —NH₂, -NH(alkyl) or halogen, and each Y₄ is, independently, -O(alkyl), —NH₂, -NH(alkyl) or -N(alkyl)₂; or
wherein one of Y₁ and Y₂ is -alkyl, -alkenyl, -alkynyl, alkylaryl, -cycloalkyl, -aryl, heteroaryl, -alkyl-Y₃ or -alkyl-C(O)Y₄, wherein each Y₃ is, independently, -O(alkyl), —NH₂, -NH(alkyl) or halogen, and each Y₄ is, independently-O(alkyl), —NH₂, -NH(alkyl) or -N(alkyl)₂, and
the other is —H; or wherein
one of Y₁ and Y₂ is -alkyl, -alkenyl, -alkynyl, alkylaryl, -cycloalkyl, -aryl, heteroaryl, -alkyl-Y₃ or -alkyl-C(O)Y₄, wherein each Y₃ is, independently, —OH, -O(alkyl), —NH₂, -NH(alkyl) or halogen, and each Y₄ is, independently, —OH, -O(alkyl), —NH₂, -NH(alkyl) or -N(alkyl)₂, and
the other is —H; or
wherein Y₁ and Y₂ are each H; or
wherein Z₁ is —CH₂CH₃ and Z₂ is —H; or
wherein Z₁ is —CH₂CH₃; Z₂ is —H; and X₁ is —H; or
wherein Z₁ is —CH₂CH₃; Z₂ is —H; and X₁ is - (alkyl) ; or
wherein one of X₁, Y₁, Y₂, Z₂, R₁, R₃ and R₄ is other than —H; or
wherein two of X₁, Y₁, Y₂, Z₂, R₁, R₃ and R₄ are other than —H; or
wherein three of X₁, Y₁, Y₂, Z₂, R₁, R₃ and R₄ are other than —H; or
wherein four of X₁, Y₁, Y₂, Z₂, R₁, R₃, and R₄ are other than —H.

9-10. (canceled)

11. The compound of claim 1 having the structure: 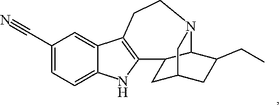 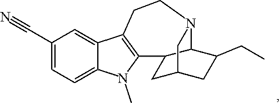 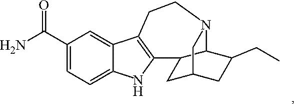 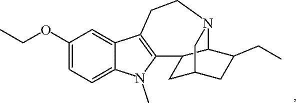 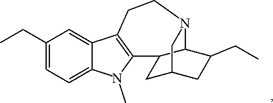 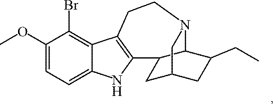 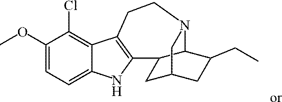 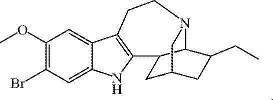
.

12. The compound of any one of claim 11 wherein
R₂ is —OCH₃ or —OCH₂CH₃.

13. The compound of claim 12 wherein R₂ is 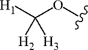
wherein at least one of H₁, H₂ or H₃ is a deuterium-enriched H site, or 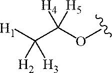
wherein at least one of H₁, H₂, H₃, H₄ or H₅ is a deuterium-enriched —H site.

14. A composition which comprises a compound which is a mixture of deuterium containing and non-deuterium containing molecules having the structure of claim 13 or a pharmaceutically acceptable salt of the compound,
wherein in the mixture the proportion of molecules having deuterium at least one of H₁, H₂, H₃ H₄ or H₅ position is substantially greater than 0.0156% of molecules in the mixture.

15. A composition which comprises a carrier and a compound having the structure of claim 13 or a pharmaceutically acceptable salt of the compound.

16. The composition of claim 14, wherein R₂ is 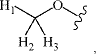
wherein at least one of H₁, H₂ or H₃ is a deuterium-enriched —H site; or
wherein each of H₁-H₃ are deuterium-enriched; or
wherein two of H₁-H₃ are deuterium-enriched; or
wherein one of H₁-H₃ is deuterium-enriched;
wherein the proportion of molecules having deuterium at each of the H₁-H₃ positions is substantially greater than 90% of molecules in the composition; or
wherein the proportion of molecules having deuterium at two of the H₁-H₃positions is substantially greater than 90% of molecules in the composition; or
wherein the proportion of molecules having deuterium at one of the H₁-H₃ positions is substantially greater than 90% of molecules in the composition; or
wherein R₂ is 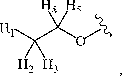
wherein at least one of H₁, H₂, H₃, H₄ or H₅ is a deuterium-enriched —H site; or
wherein each of H₁-Hₛ are deuterium-enriched; or
wherein each of H₄-H₅ are deuterium-enriched or one of H₄-H₅ is deuterium-enriched;
wherein the proportion of molecules having deuterium at each of the H₁-H₅ positions is substantially greater than 90% of molecules in the composition; or
wherein the proportion of molecules having deuterium at each of the H₄-H₅ positions is substantially greater than 90% of molecules in the composition, or the proportion of molecules having deuterium at one of the H₄-H₅ positions is substantially greater than 90% of molecules in the composition; or
wherein R₂ is 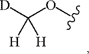 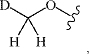 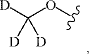 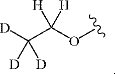 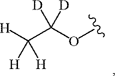 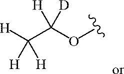 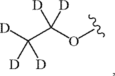
wherein D represents a deuterium-enriched —H site.

17-20. (canceled)

21. The composition of claim 15, wherein R₁ is 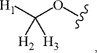
wherein at least one of H₁, H₂ or H₃ is a deuterium-enriched —H site; or
wherein each of H₁-H₃ are deuterium-enriched; or
wherein two of H₁-H₃ are deuterium-enriched; or
wherein one of H₁-H₃ is deuterium-enriched; or
wherein R₁ is 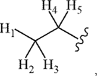
wherein at least one of H₁, H₂, H₃, H₄ or H₅ is a deuterium-enriched —H site; or
wherein each of H₁-H₅ are deuterium-enriched or each of H₁-H₃ are deuterium-enriched; or
wherein each of H₄-H₅ are deuterium-enriched or one of H₄-H₅ is deuterium-enriched; or
wherein R₂ is 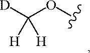 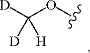 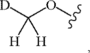 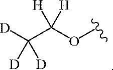 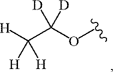 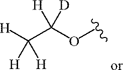 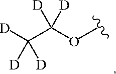
wherein D represents a deuterium-enriched —H site.

22-27. (canceled)

28. The composition of claim 15, further comprising an opioid or opiate;
wherein the opioid or opiate is morphine, hydromorphone, oxymorphone, codeine, dihydrocodeine, hydrocodone, oxycodone, nalbuphine, butorphanol, etorphine, dihydroetorphine, levorphanol, metazocine, pentazocine, meptazinol, meperidine (pethidine), fentanyl, sufentanil, alfentanil, buprenorphine, methadone, tramadol, tapentadol, mitragynine, 3-deutero-mitragynine, 7-hydroxymitragynine, 3-deutero-7-hydroxymitragynine, mitragynine pseudoindoxyl, tianeptine, 7-((3-bromo-6-methyl-5,5-dioxido-6,11-dihydrodibenzo[c,f][1,2]thiazepine-11-yl)amino)heptanoic acid, 7-((3-iodo-6-methyl-5,5-dioxido-6,11-dihydrodibenzo[c,f][1,2]thiazepine-11-yl)amino)heptanoic acid, 5-((3-bromo-6-methyl-5,5-dioxido-6,11-dihydrodibenzo[c,f][1,2]thiazepine-11-yl)amino)pentanoic acid or 5-((3-iodo-6-methyl-5,5-dioxido-6,11-dihydrodibenzo[c,f] [1,2]thiazepine-11-yl)amino)pentanoic acid.

29. (canceled)

30. A method of altering the psychological state of a subject comprising administering to the subject the compound of claim 1 comprising an effective amount of the compound, so as to thereby alter the psychological state of the subject; or
a method of enhancing the effect of psychotherapy in a subject comprising an effective amount of the compound, so as to thereby enhance the effect of the psychotherapy in the subject; or
a method of inducing wakefulness or decreasing sleepiness in a subject comprising administering to the subject an effective amount of the compound, so as to thereby induce wakefulness or decrease sleepiness in the subject;.or
a method of inducing a stimulating effect in a subject comprising administering to the subject an effective amount of the compound, so as to thereby induce a stimulating effect in the subject; or
a method of treating a subject afflicted with substance use disorder comprising administering to the subject an effective amount of the compound, so as to thereby treat the subject afflicted with the substance use disorder,
wherein the substance use disorder is opioid use disorder, alcohol use disorder or stimulant use disorder; or
a method of treating a subject afflicted with opioid withdrawal symptoms comprising administering to the subject an effective amount of the compound, so as to thereby treat the subject afflicted with the opioid withdrawal symptoms; or
a method of treating a subject afflicted with a depressive disorder, a mood disorder, an anxiety disorder, Parkinson’s disease, or traumatic brain injury comprising administering to the subject an effective amount of the compound, so as to thereby treat the subject afflicted with the depressive disorder, the mood disorder, the anxiety disorder, Parkinson’s disease or the traumatic brain injury.

31-36. (canceled)

37. A method of treating a subject afflicted with pain comprising administering to the subject the composition of claim 28 comprising an effective amount of the compound and the opioid or opiate so as to thereby treat the subject afflicted with pain.

38. The method of claim 37, wherein an effective amount of 10-1500 mg of the compound is administered to the subject.

39. A method of inhibiting serotonin transporter (SERT) and/or vesicular monoamine transporter 2 (VMAT2) in a subject comprising administering to the subject an effective amount of a compound having the structure: 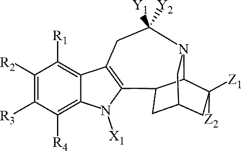
wherein
X₁ is H or alkyl;
Y₁ is H, -alkyl, -alkenyl, -alkynyl, alkylaryl, -cycloalkyl, -aryl, heteroaryl, -alkyl-Y₃ or -alkyl-C(O)Y₄, and
Y₂ is H, -alkyl, -alkenyl, -alkynyl, alkylaryl, -cycloalkyl, -aryl, heteroaryl, -alkyl-Y₃ or -alkyl-C(O)Y₄,
wherein each Y₃ is, independently, —OH, -O(alkyl), —NH₂, -NH(alkyl) or halogen, and each Y₄ is, independently, —OH, O(alkyl), —NH₂, -NH(alkyl) or -N(alkyl)₂;
Z₁ is H, -alkyl, -alkenyl, -alkynyl, alkylaryl, -aryl, heteroaryl, -alkyl-Z₃ or -alkyl-C(O)Z₄, and
Z₂ is H, -alkyl, -alkenyl, -alkynyl, alkylaryl, -aryl, heteroaryl, -alkyl-Z₃ or -alkyl-C(O)Z₄,
wherein each Z₃ is, independently, —OH, -O(alkyl), —NH₂, -NH(alkyl) or halogen, and each Z₄ is, independently, —OH, O(alkyl),NH₂, -NH(alkyl) or -N(alkyl)₂;
R₁, R₂, R₃ and R₄ are each, independently, —H, —F, —Cl, —Br, —I, —NO₂, —CN, —CF₃, —CF₂H, —OCF₃, -(alkyl), -(alkenyl), -(alkynyl), (aryl),(heteroaryl), —OH, —OAc, -O-(alkyl), -O-(alkenyl), -O-(alkynyl), -O-(aryl), -O-(heteroaryl), —SH, -S-(alkyl), -S-(alkenyl), -S-(alkynyl), -S-(aryl), -S-(heteroaryl), —NH₂, -NH-(alkyl), -NH-(alkenyl), -NH-(alkynyl), -NH-(aryl), -NH-(heteroaryl), -C(O)R₅, -S(O)R₅, -SO₂R₅, -NHSO₂R₅, -OC(O)R₅, -SC(O) R₅, -NHC(O)R₆ or -NHC(S)R₆,
wherein each R₅ is, independently, -(alkyl), -(aryl), -(heteroaryl), —OH, -O(alkyl), —NH₂, -NH(alkyl) or -N(alkyl)₂, and
wherein each R₆ is, independently, -(alkyl), -(aryl), -O-(alkyl), -S-(alkyl), -S-(aryl), —NH₂, -NH(alkyl) or -N (alkyl) ₂,
or a pharmaceutically acceptable salt thereof, so as to thereby inhibit serotonin transporter (SERT) and/or vesicular monoamine transporter 2 (VMAT2) in the subject.

40. The method of claim 39, wherein the method is for altering the psychological state of the subject; or
wherein the method is for enhancing the effect of psychotherapy in the subject; or
wherein the method is for inducing wakefulness or decreasing sleepiness in the subject; or
wherein the method is for inducing a stimulating effect in the subject; or
wherein the method is for treating the subject afflicted with substance use disorder,
wherein the substance use disorder is opioid use disorder, alcohol use disorder or stimulant use disorder; or
wherein the method is for treating the subject afflicted with opioid withdrawal symptoms; or
wherein the method is for treating the subject afflicted with a depressive disorder, a mood disorder, an anxiety disorder, Parkinson’s disease, or traumatic brain injury; or
wherein the method is for inhibiting vesicular monoamine transporter 2 (VMAT2), or inhibiting both VMAT2 and SERT;
wherein the method is for treating the subject afflicted with substance use disorder, Tardive dyskinesia (TD), Tourette syndrome, and chorea associated with Huntington’s disease.

41-43. (canceled)

44. The method of claim 39, wherein an effective amount of 10-1500 mg of the compound is administered to the subject.

45. The method of claim 44, wherein the compound has the structure: 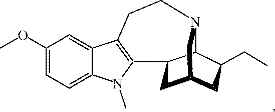 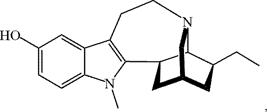 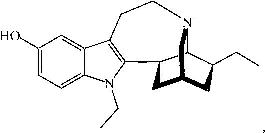 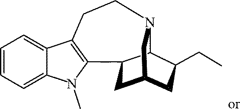 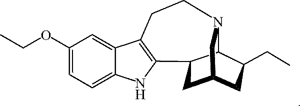
.

46. A process for producing the compound of claim 1 having the structure: 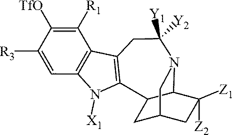
comprising
(a) contacting a compound having the structure: 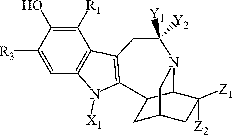
with bis (trifluoromethanesulfonyl) aniline in a first suitable solvent; and
(b) adding a base to produce the compound having the structure: 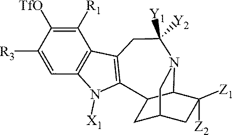
,
wherein the first suitable solvent is dichloroethane; or
wherein the base is triethylamine; or
a process for the compound having the structure: 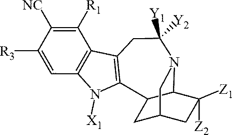
comprising
contacting a compound having the structure: 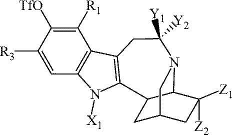
with a preformed palladium (0) catalyst in the presence of ZnCN
₂ in a first suitable solvent to produce the compound having the structure: 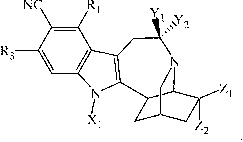
wherein the palladium (0) catalyst is Pd(PPh₃)₄; or
wherein the first suitable solvent is DMF; or
a process for producing the compound having the structure: 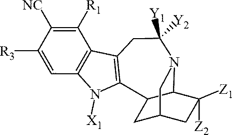
comprising
contacting a compound having the structure: 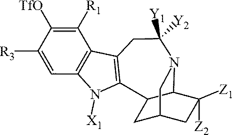
with a preformed palladium (0) catalyst in the presence of ZnCN
₂ in a first suitable solvent to produce the compound having the structure: 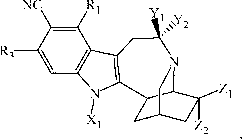
wherein the palladium (0) catalyst is Pd(PPh₃)₄; or
wherein the first suitable solvent is DMF; or
a process for producing the compound having the structure: 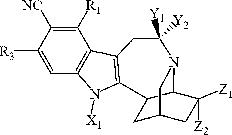
comprising
contacting a compound having the structure: 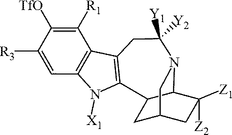
with a preformed palladium (0) catalyst in the presence of ZnCN
₂ in a first suitable solvent to produce the compound having the structure: 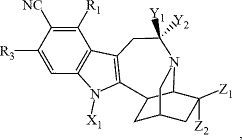
wherein the palladium (0) catalyst is Pd(PPh₃)₄; or
wherein the first suitable solvent is DMF; or
a process for producing the having the structure: 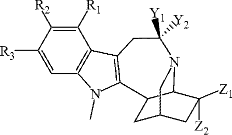
comprising
(a) contacting a compound having the structure: 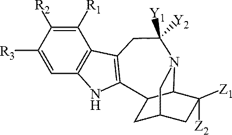
with a base in a first suitable solvent;
(b) adding a methylating reagent to produce the compound having the structure: 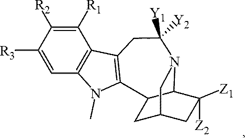
wherein the first suitable solvent is DMSO; or
wherein the base is potassium hydroxide; or
wherein the methylating reagent is iodomethane; or
a process for producing the compound having the structure: 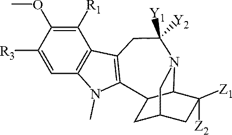
comprising
(a) contacting a compound having the structure: 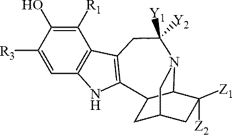
with a base in a first suitable solvent; and
(b) adding a methylating reagent to produce the compound having the structure: 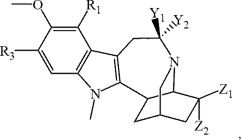
wherein the first suitable solvent is DMSO;
wherein the base is potassium hydroxide; or
wherein the methylating reagent is iodomethane; or
a process for producing the compound having the structure: 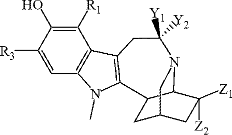
comprising
contacting a compound having the structure: 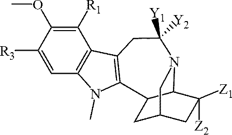
with BBr
₃ and a nucleophile in a first suitable solvent to produce the compound having the structure: 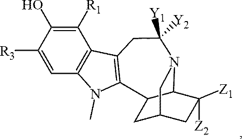
wherein the first suitable solvent is dichloromethane; or
wherein the nucleophile is EtSH; or
a process for producing the compound having the structure: 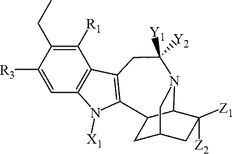
comprising
(a) contacting a compound having the structure: 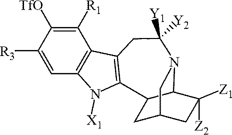
with a palladium (0) reagent in the presence of a ligand, base and CH
₃CH₂BF₃K.
(b) adding a first suitable solvent mixture to produce the compound having the structure: 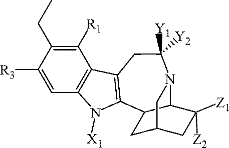
wherein the palladium (0) reagent is Pd(Oac)₂; or
wherein the ligand is RuPhos; or
wherein the base is cesium carbonate; or
wherein the first suitable solvent mixture is toluene and water.

47-57. (canceled)
